(19) United States
(12) Patent Application Publication
(10) Pub. No.: US 2025/0161228 A1
(43) Pub. Date: May 22, 2025

(54) COMPOSITIONS COMPRISING ULTRAFINE BUBBLES AND METHODS OF USING THEREOF IN A METHOD OF PRODUCING AND DELIVERING ACTIVE PHARMACEUTICAL INGREDIENTS AND OTHER DISSOLVED SOLUTES

(71) Applicant: Hydrosome IP, LLC, Westmont, IL (US)

(72) Inventors: Peter A. Kozak, Bolingbrook, IL (US); John Nicholas Jackowetz, Rochester, NY (US); Carly S. Hanson, Naperville, IL (US); Kiriako D. Tsoukalas, Munster, IN (US); Bob Jacobs, Newtown, CT (US); David C. Bom, Union, KY (US)

(21) Appl. No.: 18/946,181

(22) Filed: Nov. 13, 2024

Related U.S. Application Data
(60) Provisional application No. 63/599,695, filed on Nov. 16, 2023.
(60) Provisional application No. 63/562,417, filed on Mar. 7, 2024.

(51) Int. Cl. A61K 9/51

(52) U.S. Cl. CPC A61K 9/5192

(57) ABSTRACT

The disclosure provides compositions including ultrafine bubbles having water and gases released from solution in the water. The compositions may dissolve, surround, and/or stabilize one or more non-gaseous solutes. Methods of making and using the compositions for delivering an active pharmaceutical ingredient to cells are also provided. The methods of making the compositions including ultrafine bubbles include processes for dissolving, surrounding, and/or stabilizing non-gaseous solutes and/or active pharmaceutical ingredients with ultrafine bubbles. Methods of using the compositions to produce active pharmaceutical ingredients and/or increase yield thereof via fermentation are also provided.

### FIELD

The present disclosure generally relates to aqueous compositions that comprise soft cavitated water and/or ultrafine bubbles for use in methods of delivering active pharmaceutical ingredients and other dissolved solutes to cells.

### BACKGROUND

Ultrafine bubbles in aqueous compositions can be generated when “cavitation” occurs. There are two fundamental types of cavitation: (1) vaporous or “hard” cavitation and (2) gaseous or “soft” cavitation. Vaporous cavitation occurs when the pressure in a liquid drops below the vapor pressure of the liquid, resulting in the formation of unstable low-pressure voids or bubbles formed from vaporized particles or molecules of the liquid itself. By contrast, gaseous cavitation occurs when gases dissolved within a liquid fall out of solution with decreasing pressure, typically at pressures higher than the vapor pressure of the liquid itself-creating bubbles formed from particles or molecules of the liquid and the released gases.

Typical aqueous ultrafine bubbles are created as a result of vaporous or “hard” cavitation, when the resulting voids collapse and form shockwaves. The shockwaves facilitate the formation of ultrafine bubbles from exogenous non-dissolved gases. These ultrafine bubbles have substantially different structural, functional, and stability characteristics than ultrafine bubbles formed from “gaseous” cavitation. In aqueous compositions, ultrafine bubbles formed from “hard” cavitation (which comprise bubbles including water molecules surrounding exogenously provided gases) have substantially different physical characteristics in terms of structure, function, and stability than ultrafine bubbles formed from “soft” cavitation (which comprise bubbles including water molecules surrounding gases released from solution within the water).

With respect to aqueous compositions, it is well known that the organization of water molecules influences the stability, solubility, and bioavailability of any solutes dissolved within. As the organization of water molecules in compositions having ultrafine bubbles produced via vaporous or “hard” cavitation differs substantially from that of the organization of water molecules in compositions containing ultrafine bubbles produced via gaseous or “soft” cavitation, the stability, solubility, and bioavailability of solutes dissolved within the two compositions may be substantially different. It would be beneficial to produce aqueous compositions including water and ultrafine bubbles comprising gases released from solution in water—that is, produced via gaseous or “soft” cavitation—which compositions have improved stability, solubility, and bioavailability as compared to compositions including no ultrafine bubbles or solutions comprising or consisting of aqueous ultrafine bubbles formed via vaporous or “hard” cavitation. There also exists a need for compositions that comprise water, ultrafine bubbles comprising gases released from solution in water, and a non-gaseous solute that have improved bioavailability, solubility, and/or stability. The non-gaseous solutes with improved bioavailability have been shown to be effective in permeating cell membranes and delivering the non-gaseous solutes into cells. This ability to penetrate allows the compositions to be effective means to deliver active pharmaceutical ingredients into human cells.

Additionally, fermentation is vital for producing complex molecules that are challenging to synthesize chemically, playing a critical role in the production of antibiotics, vitamins, enzymes, and other medically significant compounds and active pharmaceutical ingredients. There also exists a need for improving production of these compounds and increasing yield thereof.

Oxygenation is a crucial aspect of bioreactor design, especially in processes involving aerobic microorganisms or cell cultures that require oxygen for metabolism and growth. The availability of oxygen can be a limiting factor in achieving optimal cell densities and production rates. Efficient oxygen transfer is essential to support the metabolic needs of cells and maintain optimal conditions for bioprocessing. In traditional bioreactor systems, oxygen is typically delivered through sparging, where air or oxygen is bubbled through the culture medium. However, challenges include mass transfer limitations, bubble coalescence, and shear stress on cells due to high gas flow rates.

Therefore, there is a need for a method to leverage the production of microbubbles and nanobubbles together to improve aeration efficiency for fermentation processes in bioreactors. The present technology is leveraged with a microbubble generator (such as a dissolved air flotation (DAF) pump or pinpoint aeration with dwell tube) for infusing the culture media with a mixture of both air microbubbles and ultrafine bubbles, increasing the oxygen transfer rate (OTR) and the oxygen uptake rate (OUR).

### SUMMARY

The present disclosure provides aqueous compositions and methods for improving the bioavailability and cell permeability of and delivering active pharmaceutical ingredients, the compositions or solutions including gaseous ultrafine bubbles (i.e., ultrafine bubbles that comprise or consist essentially of water and gases released from solution in the water and produced via gaseous or “soft” cavitation) and at least one non-gaseous solute (e.g., a cellular detoxification agent, a hydration agent, an anti-inflammatory agent, a neuroprotective agent, a neuromodulatory agent, or an anti-tumorigenic agent) dissolved within, surrounded by, and/or stabilized by the ultrafine bubbles.

The present disclosure also provides methods for increasing biomass of microbes, for producing biological compounds, and/or for increasing yields of biological compounds/active pharmaceutical ingredients via fermentation using compositions according to the disclosure herein.

In some embodiments, the water is selected from DI water, ultrapure water, tap water, groundwater (e.g., well water), surface water, and reverse osmosis water. In particular embodiments, the water is ultrapure water. In particular embodiments, the water is DI water. In some embodiments, the water is tap water.

In some embodiments, the applied compositions include water having a population of ultrafine bubbles with a median ultrafine bubble diameter of between about 2-400 nanometers. In another embodiment, the ultrafine bubbles have a median diameter of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median diameter of between about 10 to about 15 nanometers, about 15 to about 20 nanometers, or about 20 to about 25 nanometers. In other embodiments, the ultrafine bubbles have a median diameter of between about 10 to about 50 nanometers, about 20 to about 50 nanometers, about 30 to about 50 nanometers, or about 40 to about 50 nanometers. In still other embodiments, the ultrafine bubbles have a median diameter of between about 50 to about 100 nanometers. In yet further embodiments, the ultrafine bubbles have a median diameter of between about 100 to about 200 nanometers, about 150 to about 200 nanometers, about 200 to about 300 nanometers, about 250 to about 300 nanometers, or about 300 to about 400 nanometers.

In some embodiments, the ultrafine bubbles are present in the composition at a concentration of up to 10¹⁰ ultrafine bubbles/mL, as measured via nanoparticle tracking analysis (NTA), which is able to detect bubbles with diameters of 50 to 1000 nanometers. In some embodiments, the ultrafine bubbles are present in the composition at a range of 10 to 10² ultrafine bubbles/mL, 10² to 10³ ultrafine bubbles/mL, 10³ to 10⁴ ultrafine bubbles/mL, 10⁴ to 10⁵ ultrafine bubbles/mL, 10⁵ to 10⁶ ultrafine bubbles/mL, 10⁶ to 10⁷ ultrafine bubbles/mL, 10⁷ to 10⁸ ultrafine bubbles/mL, 10⁸ to 10⁹ ultrafine bubbles/mL, or 10⁹ to 10¹⁰ ultrafine bubbles/mL.

In certain embodiments in which the water of the composition is enriched via microbubble generation prior to generation of the ultrafine bubbles in the composition, the concentration of ultrafine bubbles in the resulting composition may be higher. In such embodiments, the ultrafine bubbles may be present in the composition at a range of 10¹⁰ to 10¹¹ ultrafine bubbles/mL. In some embodiments, the ultrafine bubbles are present in the composition at a range of 10¹⁰ to 10¹¹ ultrafine bubbles/mL.

In some embodiments, the water has an oxidative reduction potential from about −200 mV to about 800 mV (e.g., about −200 mV, about −195 mV, about −190 mV, about −185 mV, about −180 mV, about −175 mV, about −170 mV, about −165 mV, about −160 mV, about −155 mV, about −150 mV, about −145 mV, about −140 mV, about −135 mV, about −130 mV, about −125 mV, about −120 mV, about −115 mV, about −110 mV, about −105 mV, about −100 mV, about −95 mV, about-90 mV, about −85 mV, about −80 mV, about −75 mV, about −70 mV, about −65 mV, about −60 mV, about −55 mV, about −50 mV, about −45 mV, about −40 mV, about −35 mV, about −30 mV, about −25 mV, about −20 mV, about −15 mV, about −10 mV, about −5 mV, about 0 mV, about 5 mV, about 10 mV, about 15 mV, about 20 mV, about 25 mV, about 30 mV, about 35 mV, about 40 mV, about 45 mV, about 50 mV, about 55 mV, about 60 mV, about 65 mV, about 70 mV, about 75 mV, about 80 mV, about 85 mV, about 90 mV, about 95 mV, about 100 mV, about 105 mV, about 110 mV, about 115 mV, about 120 mV, about 125 mV, about 130 mV, about 135 mV, about 140 mV, about 145 mV, about 150 mV, about 155 mV, about 160 mV, about 165 mV, about 170 mV, about 175 mV, about 180 mV, about 185 mV, about 190 mV, about 195 mV, about 200 mV, about 205 mV, about 210 mV, about 215 mV, about 220 mV, about 225 mV, about 230 mV, about 235 mV, about 240 mV, about 245 mV, about 250 mV, about 255 mV, about 260 mV, about 265 mV, about 275 mV, about 280 mV, about 290 mV, about 295 mV, about 300 mV, about 305 mV, about 310 mV, 315 mV, 320 mV, 325 mV, 330 mV, 335 mV, 340 mV, 345 mV, 350 mV, 355 mV, 360 mV, 365 mV, 370 mV, 375 mV, 380 mV, 385 mV, 390 mV, 395 mV, 400 mV, 405 mV, 410 mV, 415 mV, 420 mV, 425 mV, 430 mV, 435 mV, 440 mV, 445 mV, 450 mV, 455 mV, 460 mV, 465 mV, 470 mV, 475 mV, 480 mV, 485 mV, 490 mV, 495 mV, 500 mV, 505 mV, 510 mV, 515 mV, 520 mV, 525 mV, 530 mV, 535 mV, 540 mV, 545 mV, 550 mV, 555 mV, 560 mV, 565 mV, 570 mV, 575 mV, 580 mV, 585 mV, 590 mV, 595 mV, about 600 mV, about 605 mV, about 610 mV, about 615 mV, about 620 mV, about 625 mV, about 630 mV, about 635 mV, about 640 mV, about 645 mV, about 650 mV, about 655 mV, about 660 mV, about 665 mV, about 670 mV, about 675 mV, about 680 mV, about 685 mV, about 690 mV, about 695 mV, about 700 mV, about 705 mV, about 710 mV, about 715 mV, about 720 mV, about 725 mV, about 730 mV, about 735 mV, about 740 mV, about 745 mV, about 750 mV, about 755 mV, about 760 mV, about 765 mV, about 770 mV, about 775 mV, about 780 mV, about 785 mV, about 790 mV, about 795 mV, or about 800 mV).

In some embodiments, the pH of the water is between about 4 to about 8 (e.g., about 4, about 5, about 6, about 7, or about 8). In some embodiments of each or any of the above- or below-mentioned embodiments, the water has a resistivity between about 17 to about 18.2 meg-ohm cm.

In some embodiments, the compositions comprise ultrafine bubbles comprising or consisting essentially of water and gases released from solution in water, wherein the ultrafine bubbles optionally dissolve, surround, and/or stabilize a non-gaseous solute, and wherein the composition has a zeta potential of between about absolute value 0 and 40. In further embodiments, the zeta potential of the composition is between about −40 mV to about 0 mV. In still further embodiments, the zeta potential of the composition is between about −40 mV to about −35 mV, about −35 mV to about −30 mV, about −30 mV to about −25 mV, about −25 mV to about-20 mV, about −20 mV to about −15 mV, about −15 mV to about −10 mV, about −10 mV to about −5 mV, about −5 mV to about 0 mV, about 0 mV to about 5 mV, about 5 mV to about 10 mV, about 10 mV to about 15 mV, about 15 mV to about 20 mV, about 20 mV to about 25 mV, about 25 mV to about 30 mV, about 30 mV to about 35 mV, or about 35 mV to about 40 mV. The inventors have surprisingly found that despite relatively low zeta potentials of the compositions (e.g., typical ultrafine bubble compositions have zeta potentials of around absolute value 30 mV, significantly higher than the absolute value zeta potentials of the compositions herein), the ultrafine bubble compositions according to the disclosure herein achieve superior stability results over ultrafine bubbles formed by alternative means with higher absolute value zeta potentials.

In some embodiments, the applied compositions include at least one non-gaseous solute. In further embodiments, the at least one non-gaseous solute is dissolved within, surrounded by, and/or stabilized by the ultrafine bubbles.

In some embodiments, the compositions comprise at least one non-gaseous solute (e.g., a solute dissolved within the composition). In further embodiments, the at least one non-gaseous solute is dissolved within, surrounded by, or stabilized by the ultrafine bubbles. In particular embodiments, the composition increases cell permeability and/or bioavailability of the at least one dissolved non-gaseous solute. In some embodiments, the at least one non-gaseous solute dissolved within or stabilized by the ultrafine bubbles has improved bioavailability relative to a solute not dissolved within or stabilized by the ultrafine bubbles. In further embodiments the at least one non-gaseous solute dissolved within or stabilized by the ultrafine bubbles has improved stability relative to a solute not dissolved within or stabilized by the ultrafine bubbles. In still further embodiments, the at least one non-gaseous solute dissolved within or stabilized by the ultrafine bubbles has improved solubility relative to a solute not dissolved within or stabilized by the ultrafine bubbles.

In some embodiments, the ultrafine bubbles (e.g., ultrafine bubbles comprising gases released from solution in water) are concentrated within the composition via rotary evaporation and/or cross flow filtration.

In some embodiments, the compositions and/or ultrafine bubbles are stable and/or exhibit biological efficacy for at least six months, for at least one year, for at least 2 years, for at least 3 years, for at least 4 years, or for at least 5 years.

In some embodiments of each or any of the above- or below-mentioned embodiments, the composition is used to deliver an active pharmaceutical ingredient. The present disclosure also provides a composition that includes ultrafine bubbles comprising or consisting essentially of water and gases released from solution in the water, wherein the ultrafine bubbles have a median ultrafine bubble diameter of between about 2 to about 400 nanometers, and wherein the ultrafine bubbles dissolve, surround, and/or stabilize a non-gaseous solute. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

In some embodiments, the composition is used for active pharmaceutical ingredient delivery. In some embodiments of each or any of the above- or below-mentioned embodiments, the composition is used to deliver at least one non-gaseous solute to the interior of a cell (e.g., a human cell). In an embodiment of each or any of the above- or below-mentioned embodiments, the at least one non-gaseous solute dissolved within or stabilized by the ultrafine bubbles comprises an active pharmaceutical ingredient. In some embodiments, the composition includes ultrafine bubbles comprising or consisting essentially of water and gases released from solution in the water, wherein the ultrafine bubbles dissolve, surround, and/or stabilize the at least one non-gaseous solute (e.g., active pharmaceutical ingredient).

In another aspect disclosed herein, a method for producing a composition comprising water and ultrafine bubbles, wherein the ultrafine bubbles are at a concentration of up to 10¹⁰ ultrafine bubbles/mL is provided. The method includes subjecting water to a combination of hydrodynamic cavitation, shear forces, and thin film boiling to produce ultrafine bubbles formed by release of dissolved gases from the water. In some embodiments, the water is selected from DI water, ultrapure water, tap water, groundwater (e.g., well water), surface water, and reverse osmosis water. In particular embodiments, the water is ultrapure water. In some embodiments, the ultrafine bubbles are present in the composition at a range of 10 to 10² ultrafine bubbles/mL, 10² to 10³ ultrafine bubbles/mL, 10³ to 10⁴ ultrafine bubbles/mL, 10⁴ to 10⁵ ultrafine bubbles/mL, 10⁵ to 10⁶ ultrafine bubbles/mL, 10⁶ to 10⁷ ultrafine bubbles/mL, 10⁷ to 10⁸ ultrafine bubbles/mL, 10⁸ to 10⁹ ultrafine bubbles/mL, or 10⁹ to 10¹⁰ ultrafine bubbles/mL.

In certain embodiments in which the water of the composition is enriched via microbubble generation prior to generation of the ultrafine bubbles in the composition, the concentration of ultrafine bubbles in the resulting composition may be higher. In such embodiments, the ultrafine bubbles may be present in the composition at a range of 10⁸ to 10¹² ultrafine bubbles/mL. In some embodiments, the ultrafine bubbles are present in the composition at a range of 10⁸ to 10⁹ ultrafine bubbles/mL, 10⁹ to 10¹⁰ ultrafine bubbles/mL, or 10¹⁰ to 10¹¹ ultrafine bubbles/mL.

In an embodiment of the method, the method further comprises dissolving at least one non-gaseous solute into the composition. In some embodiments, the at least one non-gaseous solute is dissolved within, surrounded by, and/or stabilized by the ultrafine bubbles.

In an embodiment of each or any of the above- or below-mentioned embodiments, the at least one non-gaseous solute dissolved within, surrounded by, and/or stabilized by the ultrafine bubbles comprises an active pharmaceutical ingredient.

In some embodiments, the composition is used to deliver the at least one non-gaseous solute to a cell (e.g., the interior of the cell). In some embodiments, the cell is a human cell. In particular embodiments, the human cell is one or more of a human embryonic kidney cell, epithelial intestinal cell, epidermal cell, tracheal and bronchial epithelial cell, buccal cell, nasal cell, and corneal cell, liver cell (hepatocyte), cardiomyocyte, neuronal cell (neuron), blood-brain barrier cell, muscle cell (myocyte), immune cell (e.g., macrophage, T cell), bone cell (osteoblast, osteoclast), kidney cell (proximal tubular epithelial cell), pancreatic cell (beta cell), gastrointestinal cell (enterocyte), endothelial cell, adipocyte (fat cell), skeletal muscle cell, reproductive cell (sperm, ova), cancer cell, stem cell.

In some embodiments, the composition is suitable for oral or sublingual delivery, transdermal delivery, or delivery by inhalation.

A non-gaseous solute dissolved within, surrounded by, and/or stabilized by ultrafine bubbles (e.g., ultrafine bubbles that comprise or consist essentially of water and gases released from solution in water) may be a small molecule drug, a protein, a peptide, or a combination thereof. In some embodiments, the non-gaseous solute comprises a cellular detoxification agent, a hydration agent, an anti-inflammatory agent, a neuroprotective agent, a neuromodulatory agent, or an anti-tumorigenic agent. In still other embodiments, the non-gaseous solute improves ATP production.

In some embodiments, the non-gaseous solute comprises an organic chemical, an inorganic chemical, a fat, a peptide, a sugar, a synthetic polymer including polyethylene, nylon, polypropylene, a wax, an oil, a colloid, an oligosaccharide, a polysaccharide, a protein, a fatty acid, a DNA nucleotide, a polynucleotide, an RNA polynucleotide, a pharmaceutical drug, a surfactant, a hydrogel, a hydrophilic substance catalyst, a free radical scavenger, an ion chelator, a paramagnetic substance, a magnetic field sensitive substance, a radioactive substance, a radiocontrast agent, an ultrasound contrast agent, a cerium oxide, an odorant, a perfume, a pheromone, a hormone, a cytokine, an interleukin, an antibody, a biological cell organelle, an intact biological, a fluorescent compound, a polymerase, a PCR enzyme, a catalyst, or any combination thereof.

In certain embodiments, a non-gaseous solute dissolved within, surrounded by, and/or stabilized by the ultrafine bubbles (e.g., ultrafine bubbles that comprise or consist essentially of water and gases released from solution in water) is an ion of an ionizable salt. In certain embodiments, the ion is an aluminum ion, ammonium ion, antimony ion, arsenic ion, barium ion, beryllium ion, bismuth ion, boron ion, bromide ion, cadmium ion, calcium ion, cerium ion, cesium cation, chloride ion, chromium ion, cobalt ion, copper ion, dysprosium ion, erbium ion, europium ion, fluoride ion, gadolinium ion, gallium ion, germanium ion, gold ion, hafnium ion, holmium ion, indium ion, iodine ion, iridium ion, iron ion, lanthanum ion, lead ion, lithium ion, lutetium ion, magnesium ion, manganese ion, mercury ion, molybdenum ion, neodymium ion, nickel ion, niobium ion, osmium ion, palladium ion, phosphorus ion, platinum ion, potassium ion, prascodymium ion, rhenium ion, rhodium ion, rubidium ion, ruthenium ion, samarium ion, scandium ion, selenium ion, silicon ion, silver ion, sodium ion, strontium ion, sulfate ion, tantalum ion, tellurium ion, terbium ion, thallium ion, thorium ion, thulium ion, tin ion, titanium ion, tungsten ion, vanadium ion, ytterbium ion, yttrium ion, zinc ion, or zirconium ion. In some embodiments, the ultrafine bubbles comprise about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 water molecules.

In some embodiments, the ultrafine bubbles have a median size (diameter) of between about 2 to about 400 nanometers. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 15 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

In some embodiments, the ultrafine bubbles fully dissolve, surround, and/or stabilize a non-gaseous solute. In other embodiments, the ultrafine bubbles substantially dissolve, surround, and/or stabilize a non-gaseous solute (e.g., dissolve, surround, and/or stabilize about 50%, 55%, 60%, 65%, 70%, 80%, 85%, 90%, 95% or more of the non-gaseous solute). In some embodiments, the non-gaseous solute is an active pharmaceutical ingredient.

In certain embodiments, the methods further include preparing the compositions used by pumping the water and the one or more non-gaseous solutes through a transfer pipe and a nozzle into a hollow cylinder, wherein the nozzle is located at the proximal end of the hollow cylinder. The nozzle includes an intake hole in a proximal face of the nozzle connected to the transfer pipe and one or more jet openings in a distal face of the nozzle that open into a chamber defined by the hollow cylinder. The water passing through the one or more jet openings creates a vortex of water in contact with an inner surface of the chamber. The compositions exit the hollow cylinder (containing ultrafine bubbles and the one or more non-gaseous solutes as disclosed), such that the one or more non-gaseous solutes is dissolved within, surrounded by, and/or stabilized by the ultrafine bubbles in accordance with the disclosure.

In other embodiments, the methods further include preparing the compositions used by pumping the water through a transfer pipe and a nozzle into a hollow cylinder, wherein the nozzle is located at the proximal end of the hollow cylinder. The nozzle includes an intake hole in a proximal face of the nozzle connected to the transfer pipe and one or more jet openings in a distal face of the nozzle that open into a chamber defined by the hollow cylinder. The water passing through the one or more jet openings creates a vortex of water in contact with an inner surface of the chamber. The pumped water exits the hollow cylinder (containing ultrafine bubbles as disclosed), after which the one or more non-gaseous solutes is mixed into the exited water to produce a composition in which the one or more non-gaseous solutes is dissolved within, surrounded by, and/or stabilized by ultrafine bubbles in accordance with this disclosure.

In an embodiment, the composition or solution is stable for at least about 2 years. In some embodiments, the ultrafine bubbles are stable for about 2 years, about 2.5 years, about 3 years, about 4 years, about 5 years, about 6 years, about 7 years, about 8 years, about 9 years, or about 10 years. In some embodiments, the ultrafine bubbles are stable for a period in excess of 10 years.

In other embodiments, a non-gaseous solute dissolved within, surrounded by, and/or stabilized by ultrafine bubbles has improved bioavailability by virtue of its ability to access an intracellular space. In still other embodiments, the solute dissolved within, surrounded by, and/or stabilized by ultrafine bubbles has improved bioavailability by virtue of its ability to access specific plant or animal tissue types, such as root or leaf tissue in a plant, or skin or internal organ tissues in an animal. In yet other embodiments, an ultrafine bubble comprising or consisting essentially of water and gases released from solution in water and gases released from solution in the ultrapure water has improved bioavailability relative to an ultrafine bubble that does not comprise ultrapure water.

In some embodiments, the compositions including ultrafine bubbles and dissolved, surrounded, and/or stabilized non-gaseous solutes have improved bioavailability relative to naturally occurring water and dissolved solutes. In some embodiments, the ultrafine bubbles and dissolved, surrounded, and/or stabilized non-gaseous solutes provided herein render an otherwise unavailable non-gaseous solute bioavailable, in which case the disclosure provides improved bioavailability of the solute relative to the non-gaseous solute that is not dissolved by, surrounded by, and/or stabilized by ultrafine bubbles. In other embodiments, the ultrafine bubbles comprising or consisting essentially of water and gases released from solution in water, wherein the ultrafine bubbles dissolve, surround, and/or stabilize a non-gaseous solute improve bioavailability of the solute by about 10%, about 20%, about 30%, about 40%, about 50%, about 60%, about 70%, about 80%, or about 90% relative to the solute that is not dissolved within, surrounded by, and/or stabilized by ultrafine bubbles. In further embodiments, the ultrafine bubbles comprising or consisting essentially of water and gases released from solution in water and dissolved, surrounded, and/or stabilized non-gaseous solutes improve bioavailability of the solute by about 1%, about 2%, about 3%, about 4%, about 5%, about 6%, about 7%, about 8%, or about 9%.

In some embodiments, the compositions or solutions including ultrafine bubbles and a solute dissolved within, surrounded by, and/or stabilized by ultrafine bubbles have improved solubility relative to compositions or solutions including the solute that is not dissolved within, surrounded by, and/or stabilized by ultrafine bubbles. In other embodiments, a solute dissolved within, surrounded by, and/or stabilized by ultrafine bubbles comprises a drug with increased solubility, improved pharmacokinetics, and/or increased bioavailability. As such, embodiments of the disclosure have applications where improved solubility, pharmacokinetics, and/or bioavailability is desired, for example, without limitation, in medical products, patient care, medical research, medical testing, medical equipment, cell culture, and surgical procedures.

The disclosure also provides pharmaceutical compositions including ultrafine bubbles where the ultrafine bubbles have a median ultrafine bubble diameter of between about 2 to about 400 nanometers and one or more active pharmaceutical ingredients dissolved within, surrounded by, and/or stabilized by the ultrafine bubbles. In some embodiments, the active pharmaceutical ingredient is selected from a small molecule drug, a protein, a peptide, or a combination thereof. In other embodiments, the pharmaceutical ingredient is a cellular detoxification agent, a hydration agent, an anti-inflammatory agent, a neuroprotective agent, a neuromodulatory agent, or an anti-tumorigenic agent.

In some embodiments, the disclosure provides compositions or solutions used for delivering a therapeutic agent, medicament, drug, or the like, to a subject in need thereof. In some embodiments, an isotonic saline dissolved within, surrounded by, and/or stabilized by ultrafine bubbles is used for improving a drug for intravenous use in a mammal. In certain embodiments, a drug dissolved within, surrounded by, and/or stabilized by ultrafine bubbles is used to increase solubility of the drug. In certain embodiments, an oral drug dissolved within, surrounded by, and/or stabilized by ultrafine bubbles increases bioavailability of the oral drug. Furthermore, in certain embodiments, a drug dissolved within, surrounded by, and/or stabilized by ultrafine bubbles is used as a means for increasing potency of the drug.

In some embodiments, the compositions and solutions of the disclosure are suitable for oral or sublingual delivery, transdermal delivery, or delivery by inhalation (e.g., by nasal inhalation).

In some embodiments of each or any of the above- or below-mentioned embodiments, the ultrafine bubbles comprise or consist essentially of ultrapure water having an oxidative reduction potential about −200 to about 800 mV (e.g., from about −200 mV to about 800 mV (e.g., about −200 mV, about −195 mV, about −190 mV, about −185 mV, about −180 mV, about −175 mV, about −170 mV, about −165 mV, about −160 mV, about −155 mV, about −150 mV, about −145 mV, about −140 mV, about −135 mV, about −130 mV, about −125 mV, about −120 mV, about −115 mV, about −110 mV, about −105 mV, about −100 mV, about −95 mV, about −90 mV, about −85 mV, about −80 mV, about −75 mV, about −70 mV, about −65 mV, about −60 mV, about −55 mV, about-50 mV, about −45 mV, about −40 mV, about −35 mV, about −30 mV, about −25 mV, about −20 mV, about −15 mV, about −10 mV, about −5 mV, about 0 mV, about 5 mV, about 10 mV, about 15 mV, about 20 mV, about 25 mV, about 30 mV, about 35 mV, about 40 mV, about 45 mV, about 50 mV, about 55 mV, about 60 mV, about 65 mV, about 70 mV, about 75 mV, about 80 mV, about 85 mV, about 90 mV, about 95 mV, about 100 mV, about 105 mV, about 110 mV, about 115 mV, about 120 mV, about 125 mV, about 130 mV, about 135 mV, about 140 mV, about 145 mV, about 150 mV, about 155 mV, about 160 mV, about 165 mV, about 170 mV, about 175 mV, about 180 mV, about 185 mV, about 190 mV, about 195 mV, or about 200 mV, about 205 mV, about 210 mV, about 215 mV, about 220 mV, about 225 mV, about 230 mV, about 235 mV, about 240 mV, about 245 mV, about 250 mV, about 255 mV, about 260 mV, about 265 mV, about 275 mV, about 280 mV, about 290 mV, about 295 mV, about 300 mV, about 305 mV, about 310 mV, 315 mV, 320 mV, 325 mV, 330 mV, 335 mV, 340 mV, 345 mV, 350 mV, 355 mV, 360 mV, 365 mV, 370 mV, 375 mV, 380 mV, 385 mV, 390 mV, 395 mV, 400 mV, 405 mV, 410 mV, 415 mV, 420 mV, 425 mV, 430 mV, 435 mV, 440 mV, 445 mV, 450 mV, 455 mV, 460 mV, 465 mV, 470 mV, 475 mV, 480 mV, 485 mV, 490 mV, 495 mV, 500 mV, 505 mV, 510 mV, 515 mV, 520 mV, 525 mV, 530 mV, 535 mV, 540 mV, 545 mV, 550 mV, 555 mV, 560 mV, 565 mV, 570 mV, 575 mV, 580 mV, 585 mV, 590 mV, 595 mV, about 600 mV, about 605 mV, about 610 mV, about 615 mV, about 620 mV, about 625 mV, about 630 mV, about 635 mV, about 640 mV, about 645 mV, about 650 mV, about 655 mV, about 660 mV, about 665 mV, about 670 mV, about 675 mV, about 680 mV, about 685 mV, about 690 mV, about 695 mV, about 700 mV, about 705 mV, about 710 mV, about 715 mV, about 720 mV, about 725 mV, about 730 mV, about 735 mV, about 740 mV, about 745 mV, about 750 mV, about 755 mV, about 760 mV, about 765 mV, about 770 mV, about 775 mV, about 780 mV, about 785 mV, about 790 mV, about 795 mV, or about 800 mV).

In still further embodiments, the pH of the water is between about 4 to about 8 (e.g., about 4, about 5, about 6, about 7, or about 8). In some embodiments of each or any of the above- or below-mentioned embodiments, the composition or solution is used in the method to deliver a nutrient solute to the interior of a cell (e.g., an animal cell).

The present disclosure also provides a method of using a composition or solution that includes ultrafine bubbles that comprise or consist essentially of water molecules surrounding the gases released from solution in the water dissolving, surrounding, and/or stabilizing a solute (e.g., active pharmaceutical ingredient), wherein the ultrafine bubbles have a median diameter of between about 2 to about 400 nanometers, and wherein the ultrafine bubbles have improved bioavailability relative to a composition or a solution that does not include ultrafine bubbles that comprise or consist essentially of water molecules surrounding the gases released from solution in the water. In some embodiments, the ultrafine bubbles have a median of about 150 to about 300 water molecules per ultrafine bubble. In other embodiments, the ultrafine bubbles have a median of about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 water molecules per ultrafine bubble.

The present disclosure also provides methods for improving the bioavailability of a solute (e.g., an active pharmaceutical ingredient). In certain embodiments, the methods comprise dissolving the solute in water and dissolving/surrounding/stabilizing the solute with ultrafine bubbles, wherein the ultrafine bubbles are between about 2 to about 400 nanometers in median diameter. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

The present disclosure also provides methods for dissolving, surrounding, and/or stabilizing a solute, in water comprising mixing the solute with water and dissolving/surrounding/stabilizing the solute with ultrafine bubbles having a median diameter of between about 2 to about 400 nanometers. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

The present disclosure also provides methods for increasing biomass of microbes which produce biological compounds via fermentation by using compositions in accordance with the disclosure herein, methods for producing biological compounds using the compositions herein with microbes, and methods for increasing yields of biological compounds produced via microbial fermentation using the compositions herein.

The present disclosure also provides a method for enhancing viability and proliferation of sensitive cell lines, including stem cells, by reconstituting cell culture media with ultrafine bubble suspension, particularly under conditions that are typically suboptimal for cell survival. The ultrafine bubble suspension may act as an enhancer for growth factors, such as IL4 and GM-CSF, in the culture of stem cells, by providing a supportive environment that significantly improves cell viability compared to standard laboratory-grade water. Additionally, the present method utilizes ultrafine bubble suspension for culturing stem cells at densities previously deemed too high for survival, thereby enabling more efficient and effective research and therapeutic applications.

The present disclosure also provides methods for leveraging the production of microbubbles and nanobubbles together specifically to improve aeration efficiency for fermentation processes in bioreactors.

### BRIEF DESCRIPTION OF THE FIGURES

FIG. 1 shows a diagram of a system (101) and a method for making compositions including water and ultrafine bubbles in accordance with embodiments of the disclosure.

FIG. 2 shows a graph of intracellular zinc content in HEK cells treated with a zinc-containing composition or ZnSO₄ control solution.

FIG. 3 shows a graph representing cell viability of TF-1 cells in a water composition according to the present disclosure compared with standard laboratory grade water.

FIG. 4 shows a graph representing enhanced cell viability of TF-1 cells in a water composition according to the present disclosure compared with standard laboratory grade water.

FIG. 5 is a diagram illustrating how a microbubble and a nanobubble generator are incorporated into a bioreactor system.

FIG. 6 illustrates a flowchart depicting how microbubbles and nanobubbles facilitate oxygen delivery to cell culture media and directly to cells, respectively.

FIG. 7 shows a graph representing the effects of growth of E. Coli with and without ultrafine bubbles according to the present disclosure.

### DETAILED DESCRIPTION

The present disclosure provides compositions and methods for using the compositions and solutions (e.g., aqueous compositions) that include ultrafine bubbles comprising or consisting essentially of water and gases released from solution in the water, and a non-gaseous solute. The inventors have surprisingly found that compositions according to the disclosure herein can be applied to delivering active pharmaceutical ingredients into cells. Indeed, the inventors have surprisingly discovered that aqueous compositions that comprise a low concentration of ultrafine bubbles (e.g., at a concentration of up to 10⁸ ultrafine bubbles/mL) exert improved/increased bioavailability, solubility, permeability with respect to biological membranes, and/or stability than previously anticipated, perhaps even as compared to compositions that comprise a higher concentration of ultrafine bubbles (e.g., more than 10⁸ ultrafine bubbles/mL).

The ultrafine bubbles may be used to dissolve, surround, and/or stabilize nutrients, foods, pharmaceuticals, biologic drugs, biotechnology products, inorganic or organic chemicals. Additionally, in some embodiments, the ultrafine bubbles may be used to dissolve, surround, and/or stabilize a solute for human and/or animal nutrition. In other embodiments, the ultrafine bubbles may be used as a means for creating oral drug formulations with improved drug pharmacokinetics, higher drug bioavailability, increased drug safety and/or higher selective potency. Further, in other embodiments, the ultrafine bubbles may be used to dissolve, surround, and/or stabilize a drug or a biotechnology product.

In some embodiments, the compositions and methods can be used to increase bioavailability and cell permeability of active pharmaceutical ingredients. The methods include administering compositions or solutions in accordance with the disclosure herein to deliver active pharmaceutical ingredients to cells.

Aqueous compositions are suitable for oral or sublingual delivery, transdermal delivery, or delivery by inhalation, and such compositions include non-gaseous solutes (e.g., a cellular detoxification agent, a hydration agent, an anti-inflammatory agent, a neuroprotective agent, a neuromodulatory agent, or an anti-tumorigenic agent), and the embodiments relate more particularly to use of such compositions to increase bioavailability or permeability of the orally administered or ingested non-gaseous solute.

The ultrafine bubbles may comprise or consist essentially of water and gases released from solution in the water. The ultrafine bubbles may be used advantageously to dissolve, surround, and/or stabilize a non-gaseous solute (e.g., an active pharmaceutical ingredient) and used to deliver the solute across a cell membrane of a cell (e.g., a human cell) to exert its effect. As such, the disclosed compositions and solutions provide surprising and unexpected advantages in increasing cell permeability based, for example, on the improved bioavailability, solubility, and/or stability of ultrafine bubbles comprising or consisting essentially of water and gases released from solution in the water, and non-gaseous solutes included within the composition including the ultrafine bubbles.

Also provided by the present disclosure are methods for making aqueous ultrafine bubbles (e.g., from ultrapure water), including methods for dissolving a solute (e.g., an active pharmaceutical ingredient) in an aqueous composition including ultrafine bubbles comprising or consisting essentially of water and gases released from solution in the water. The ultrafine bubbles comprising or consisting essentially of water and gases released from solution in the water may be used to dissolve, stabilize, and/or surround solutes (e.g., an active pharmaceutical ingredient). In certain embodiments of the methods, the ultrafine bubbles in the composition have a median size of between about 2 to about 400 nanometers. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

In some embodiments, the aqueous compositions including ultrafine bubbles comprising or consisting essentially of water and gases released from solution in the water have improved bioavailability relative to naturally occurring water, and relative to compositions including ultrafine bubbles not formed via gaseous cavitation. In some embodiments, the ultrafine bubbles comprising or consisting essentially of water and gases released from solution in the water improve bioavailability of the water by about 10%, about 20%, about 30%, about 40%, about 50%, about 60%, about 70%, about 80%, or about 90% relative to naturally occurring water, and/or relative to compositions including ultrafine bubbles not formed via gaseous cavitation. In further embodiments, the ultrafine bubbles comprising or consisting essentially of water and dissolved/surrounded/stabilized solutes improve bioavailability of the water by about 1%, about 2%, about 3%, about 4%, about 5%, about 6%, about 7%, about 8%, or about 9% relative to naturally occurring water, and/or relative to compositions including ultrafine bubbles not formed via gaseous cavitation.

The present disclosure also provides compositions and solutions used in the methods wherein 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the composition or solution includes ultrafine bubbles comprising or consisting essentially of water and gases released from solution in the water and a solute, wherein the ultrafine bubbles dissolve, surround, and/or stabilize the solute.

In some embodiments of the methods, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ultrafine bubbles comprising or consisting essentially of water and gases released from solution in the water in the composition or solution dissolve, surround, and/or stabilize the solute.

In some embodiments of the methods, one or more of the solutes is present at a concentration of from about 1 mg/L to about 1000 mg/L of composition according to the disclosure herein. Compositions and solutions used in the methods herein include the one or more solutes at a concentration of about 1 mg/L, about 2 mg/L, about 3 mg/L, about 4 mg/L, about 5 mg/L, about 10 mg/L, about 20 mg/L, about 30 mg/L, about 40 mg/L, about 50 mg/L, about 60 mg/L, about 70 mg/L, about 80 mg/L, about 90 mg/L, about 100 mg/L, about 110 mg/L, about 120 mg/L, about 130 mg/L, about 140 mg/L, about 150 mg/L, about 160 mg/L, about 170 mg/L, about 180 mg/L, about 190 mg/L, about 200 mg/L, about 210 mg/L, about 220 mg/L, about 230 mg/L, about 240 mg/L, about 250 mg/L, about 260 mg/L, about 270 mg/L, about 280 mg/L, about 290 mg/L, about 300 mg/L, about 310 mg/L, about 320 mg/L, about 330 mg/L, about 340 mg/L, about 350 mg/L, about 360 mg/L, about 370 mg/L, about 380 mg/L, about 390 mg/L, about 400 mg/L, about 410 mg/L, about 420 mg/L, about 430 mg/L, about 440 mg/L, about 450 mg/L, about 460 mg/L, about 470 mg/L, about 480 mg/L, about 490 mg/L, about 500 mg/L, about 510 mg/L, about 520 mg/L, about 530 mg/L, about 540 mg/L, about 550 mg/L, about 560 mg/L, about 570 mg/L, about 580 mg/L, about 590 mg/L, about 600 mg/L, about 610 mg/L, about 620 mg/L, about 630 mg/L, about 640 mg/L, about 650 mg/L, about 660 mg/L, about 670 mg/L, about 680 mg/L, about 690 mg/L, about 700 mg/L, about 710 mg/L, about 720 mg/L, about 730 mg/L, about 740 mg/L, about 750 mg/L, about 760 mg/L, about 770 mg/L, about 780 mg/L, about 790 mg/L, about 800 mg/L, about 810 mg/L, about 820 mg/L, about 830 mg/L, about 840 mg/L, about 850 mg/L, about 860 mg/L, about 870 mg/L, about 880 mg/L, about 890 mg/L, about 900 mg/L, about 910 mg/L, about 920 mg/L, about 930 mg/L, about 940 mg/L, about 950 mg/L, about 960 mg/L, about 970 mg/L, about 980 mg/L, about 990 mg/L, or about 1000 mg/L.

In some embodiments, the at least one non-gaseous solute is dissolved within, surrounded by, and/or stabilized by the ultrafine bubbles. In some embodiments, the composition increases cell permeability and/or bioavailability of the at least one dissolved non-gaseous solute. In some embodiments, the at least one non-gaseous solute is present at a concentration of 0.1-30% by weight of the composition. In other embodiments, the at least one non-gaseous solute is present at a concentration of 0.1-1% by weight of the composition, 1-2% by weight of the composition, 2-3% by weight of the composition, 3-4% by weight of the composition, 4-5% by weight of the composition, 5-6% by weight of the composition, 6-7% by weight of the composition, 7-8% by weight of the composition, 8-9% by weight of the composition, 9-10% by weight of the composition, 10-11% by weight of the composition, 11-12% by weight of the composition, 12-13% by weight of the composition, 13-14% by weight of the composition, 14-15% by weight of the composition, 15-16% by weight of the composition, 16-17% by weight of the composition, 17-18% by weight of the composition, 18-19% by weight of the composition, 19-20% by weight of the composition, 20-21% by weight of the composition, 21-22% by weight of the composition, 22-23% by weight of the composition, 23-24% by weight of the composition, 24-25% by weight of the composition, 25-26% by weight of the composition, 26-27% by weight of the composition, 27-28% by weight of the composition, 28-29% by weight of the composition, or 29-30% by weight of the composition.

In some embodiments, the at least one non-gaseous solute is present at a low concentration of 0.1-10% by weight of the composition. In other embodiments, the at least one non-gaseous solute is present at a concentration of 0.1-0.5% by weight of the composition, 0.5-1% by weight of the composition, 1-1.5% by weight of the composition, 1.5-2% by weight of the composition, 2-2.5% by weight of the composition, 2.5-3% by weight of the composition, 3-3.5% by weight of the composition, 3.5-4% by weight of the composition, 4-4.5% by weight of the composition, 4.5-5% by weight of the composition, 5-5.5% by weight of the composition, 5.5-6% by weight of the composition, 6-6.5% by weight of the composition, 6.5-7% by weight of the composition, 7-7.5% by weight of the composition, 7.5-8% by weight of the composition, 8-8.5% by weight of the composition, 8.5-9% by weight of the composition, 9-9.5% by weight of the composition, or 9.5-10% by weight of the composition.

In some embodiments, the at least one non-gaseous solute is present at a medium concentration of 10-30% by weight of the composition. In other embodiments, the at least one non-gaseous solute is present at a concentration of 10-10.5% by weight of the composition, 10.5-11% by weight of the composition, 11-11.5% by weight of the composition, 11.5-12% by weight of the composition, 12-12.5% by weight of the composition, 12.5-13% by weight of the composition, 13-13.5% by weight of the composition, 13.5-14% by weight of the composition, 14-14.5% by weight of the composition, 14.5-15% by weight of the composition, 15-15.5% by weight of the composition, 15.5-16% by weight of the composition, 16-16.5% by weight of the composition, 16.5-17% by weight of the composition, 17-17.5% by weight of the composition, 17.5-18% by weight of the composition, 18-18.5% by weight of the composition, 18.5-19% by weight of the composition, 19-19.5% by weight of the composition, 19.5-20% by weight of the composition, 20-20.5% by weight of the composition, 20.5-21% by weight of the composition, 21-21.5% by weight of the composition, 21.5-22% by weight of the composition, 22-22.5% by weight of the composition, 22.5-23% by weight of the composition, 23-23.5% by weight of the composition, 23.5-24% by weight of the composition, 24-24.5% by weight of the composition, 24.5-25% by weight of the composition, 25-25.5% by weight of the composition, 25.5-26% by weight of the composition, 26-26.5% by weight of the composition, 26.5-27% by weight of the composition, 27-27.5% by weight of the composition, 27.5-28% by weight of the composition, 28-28.5% by weight of the composition, 28.5-29% by weight of the composition, 29-29.5% by weight of the composition, or 29.5-30% by weight of the composition.

In some embodiments, the at least one non-gaseous solute is present at a high concentration of 30-95% by weight of the composition. In other embodiments, the at least one non-gaseous solute is present at a concentration of 30-35% by weight of the composition, 35-40% by weight of the composition, 40-45% by weight of the composition, 45-50% by weight of the composition, 50-55% by weight of the composition, 55-60% by weight of the composition, 60-65% by weight of the composition, 65-70% by weight of the composition, 70-75% by weight of the composition, 75-80% by weight of the composition, 80-85% by weight of the composition, 85-90% by weight of the composition, or 90-95% by weight of the composition.

In some embodiments, the composition is a preparation for oral delivery of an active pharmaceutical ingredient; i.e., an oral formulation. In some embodiments, the oral formulation is in tablet or capsule form. In some embodiments, the at least one non-gaseous solute is an active pharmaceutical ingredient. In particular embodiments, the active pharmaceutical ingredient is present within the composition at a concentration of about 0.5% to about 90% by weight of the composition. In other embodiments, the at least one active pharmaceutical ingredient is present at a concentration of 0.5-1.0% by weight of the composition, 1.0-5.0% by weight of the composition, 5.0-10.0% by weight of the composition, 10.0-15.0% by weight of the composition, 15.0-20.0% by weight of the composition, 20-30% by weight of the composition, 30-40% by weight of the composition, 40-50% by weight of the composition, 50-60% by weight of the composition, 60-70% by weight of the composition, 70-80% by weight of the composition, or 80-90% by weight of the composition.

In some embodiments, the composition is a preparation for topical delivery of an active pharmaceutical ingredient; i.e., topical preparation. In some embodiments, the topical preparation is a cream or an ointment. In some embodiments, the at least one non-gaseous solute is an active pharmaceutical ingredient. In particular embodiments, the active pharmaceutical ingredient is present within the composition at a concentration of about 0.1% to about 5.0% by weight of the composition. In other embodiments, the at least one active pharmaceutical ingredient is present at a concentration of 0.1-0.5% by weight of the composition, 0.5-1.0% by weight of the composition, 1.0-1.5% by weight of the composition, 1.5-2.0% by weight of the composition, 2.0-2.5% by weight of the composition, 2.5-3.0% by weight of the composition, 3.0-3.5% by weight of the composition, 3.5-4.0% by weight of the composition, 4.0-4.5% by weight of the composition, or 4.5-5.0% by weight of the composition.

In some embodiments, the composition is a preparation for inhalation delivery of an active pharmaceutical ingredient; i.e., an inhalable formulation. In some embodiments, the inhalable formulation is an aerosol or a powder. In some embodiments, the at least one non-gaseous solute is an active pharmaceutical ingredient. In particular embodiments, the active pharmaceutical ingredient is present within the composition at a concentration of about 0.1% to about 10% by weight of the composition. In other embodiments, the at least one active pharmaceutical ingredient is present at a concentration of 0.1-0.5% by weight of the composition, 0.5-1.0% by weight of the composition, 1.0-1.5% by weight of the composition, 1.5-2.0% by weight of the composition, 2.0-2.5% by weight of the composition, 2.5-3.0% by weight of the composition, 3.0-3.5% by weight of the composition, 3.5-4.0% by weight of the composition, 4.0-4.5% by weight of the composition, 4.5-5.0% by weight of the composition, 5.0-5.5% by weight of the composition, 5.5-6.0% by weight of the composition, 6.0-6.5% by weight of the composition, 6.5-7.0% by weight of the composition, 7.0-7.5% by weight of the composition, 7.5-8.0% by weight of the composition, 8.0-8.5% by weight of the composition, 8.5-9.0% by weight of the composition, 9.0-9.5% by weight of the composition, or 9.5-10.0% by weight of the composition.

In some embodiments, the composition is a preparation for injection of an active pharmaceutical ingredient; i.e., an injectable formulation. In some embodiments, the injectable formulation is configured for intravenous, intramuscular, and/or subcutaneous injection. In some embodiments, the at least one non-gaseous solute is an active pharmaceutical ingredient. In particular embodiments, the active pharmaceutical ingredient is present within the composition at a concentration of about 1.0% to about 50% by weight of the composition. In other embodiments, the at least one active pharmaceutical ingredient is present at a concentration of 1.0-5.0% by weight of the composition, 5.0-10.0% by weight of the composition, 10.0-15.0% by weight of the composition, 15.0-20.0% by weight of the composition, 20.0-25.0% by weight of the composition, 25.0-30.0% by weight of the composition, 30.0-35.0% by weight of the composition, 35.0-40.0% by weight of the composition, 40.0-45.0% by weight of the composition, or 45.0-50.0% by weight of the composition.

In some embodiments of the methods set forth herein, cells are exposed to a composition with an active pharmaceutical ingredient concentration of at least 5 micromolar, at least 10 micromolar, at least 15 micromolar, at least 20 micromolar, at least 25 micromolar, at least 30 micromolar, at least 35 micromolar, at least 40 micromolar, at least 45 micromolar, at least 50 micromolar, at least 55 micromolar, at least 60 micromolar, at least 65 micromolar, at least 70 micromolar, at least 75 micromolar, at least 80 micromolar, at least 85 micromolar, or at least 90 micromolar.

In other embodiments, the composition increases intracellular uptake of an active pharmaceutical ingredient by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, or at least 140%.

In other embodiments, cells barrier integrity is reduced to at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

Without being bound by theory, it is believed the compositions as disclosed herein provide for improved bioavailability, solubility, and/or stability of the ultrafine bubbles comprising or consisting essentially of water and gases released from solution in water, as well as improved bioavailability, solubility, and/or stability of any dissolved solutes because the ultrafine bubbles are produced from “soft” or gaseous cavitation rather than “hard” or vaporous cavitation processes. The disclosed ultrafine bubbles are believed to be (a) nucleated in the low-pressure vicinity surrounding the cavitation core, (b) sheared-off bubbles from the cavitation core itself, or (c) produced via low pressure/room temperature boiling at the core surface, such that, in the presence of turbulence and high shear stresses near the core, ultrafine bubbles are broken into smaller ultrafine bubbles through deformation (due to drag forces). The resulting compositions incorporating such ultrafine bubbles exhibit improved efficacy for dissolving solutes, even at concentrations of 10⁷ ultrafine bubbles/mL and below. Such compositions also exhibit enhanced stability over other solutions incorporating ultrafine bubbles or ultrafine bubbles produced via other means, as they can be concentrated by several orders of magnitude via rotary evaporation or crossflow filtration without ultrafine bubble loss or solute dissolution, and can even remain bottled for up to 10 years without loss of ultrafine bubble concentration or dissolution of solutes.

In cases where numerical values are indicated in the context of the present disclosure, the skilled person will understand that the technical effect of the feature in question is ensured within an interval of accuracy, which typically encompasses a deviation of the numerical value given of ±10%, and preferably of ±5%. At the very least, and not as an attempt to limit the application of the doctrine of equivalents to the scope of the claims, each numerical parameter should at least be construed in light of the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

Further definitions of terms will be given in the following in the context of which the terms are used. The following terms or definitions are provided solely to aid in the understanding of the invention. These definitions should not be construed to have a scope less than understood by a person of ordinary skill in the art.

As used herein, an “ultrafine bubble” refers to an assembly of water molecules, with a diameter less than one micron, bonded with or otherwise associated with one another by electrostatic forces, such as hydrogen bonding, ionic bonding, van der Waals forces, or the like, surrounding gases (e.g., gases released from solution in water). In some cases according to the disclosure, an ultrafine bubble further comprises a non-gaseous solute associated with the water molecules and dissolved within, surrounded by, and/or stabilized by the ultrafine bubble.

As used herein, a “solute” means a substance or particle that is fully or partially dissolved in water. In embodiments, a solute of the disclosure is dissolved within, surrounded by, and/or stabilized by ultrafine bubbles of the disclosure. A solute according to the disclosure comprises, without limitation, a plant nutrient, an ion, a polar or non-polar substance, a liquid, a solid, a lipid, a protein, a peptide, a nucleic acid, an organic compound, an inorganic compound, or any combination thereof.

As used herein, “ultrapure water” means water prepared according to one or more of the described embodiments of the disclosure. In particular, ultrapure water refers to water prepared by methods and processes disclosed herein, or water characterized as being completely free of (e.g., does not contain any detectable amount), or substantially free of (e.g., 70%, 80%, 90%, or 95% free of), one or more impurities or contaminants.

As used herein, “bioavailability” refers to the physiological availability of a given amount of a solute as distinct from its chemical potency. For example, bioavailability refers to the proportion of an administered solute that is absorbed into the tissues of a cell (e.g., a human or animal cell). Bioavailability also refers to the ability of an ultrafine bubble, solute, particle, dissolved solute, or combination thereof, to access a biological target, e.g., by crossing a biological membrane or by interacting with a biological receptor or other binding partner.

### Compositions Comprising Ultrafine Bubbles

The disclosure provides methods using compositions and solutions comprising ultrafine bubbles comprising or consisting essentially of water and gases released from solution in the water, and a non-gaseous solute dissolved within, surrounded by, and/or stabilized by the ultrafine bubbles. The ultrafine bubble may have a median ultrafine bubble size of between about 2 to about 400 nanometers or a median of about 10 to about 500 water molecules per ultrafine bubble.

In some embodiments of the methods herein, ultrapure water of the disclosure comprises water substantially free of or completely free of contaminants (e.g., an impurity). As used herein, a contaminant is a foreign substance not intentionally added to the ultrapure water produced according to the disclosure. Thus, ultrapure water substantially free of contaminants contains undetectable levels/amounts of, for example, the following contaminants: (a) pathogenic bacteria (e.g., fecal coliform), viruses (e.g., hepatitis viruses, hemorrhagic viruses, retroviruses such as AIDS virus), fungi, mycoplasm, protozoa, prokaryotes, protists, parasites, microorganisms causing infectious diseases, and their spores, eggs, DNA, RNA, or related reproductive constituents, prions, (b) toxic biochemicals including toxic proteins, lipids, carbohydrates, and toxic nucleic acids; (c) toxic inorganic chemicals (soluble and insoluble in water and including toxic heavy metals) and their particles; (d) toxic organic chemicals (soluble and insoluble in water and including pesticides) and their particles; (c) non-water organic liquids (miscible and immiscible); (f) radioactive minerals, or (g) toxic gases including ammonia, arsenic pentafluoride, arsine, bis(trifluoromethyl) peroxide, boron tribromide, boron trichloride, boron trifluoride, bromine, bromine chloride, boromethane, carbon monoxide, chlorine, chlorine pentafluoride, chlorine trifluoride, chloropicrin, cyanogen, cyanogen chloride, diazomethane, diborane, dichloroacetylene, dichlorosilane, fluorine, formaldehyde, germane, hexylethyl tetraphosphate, hydrogen azide, hydrogen cyanide, hydrogen selenide, hydrogen sulfide, hydrogen telluride, nickel tetracarbonyl, nitrogen dioxide, osmium tetroxide, oxygen difluoride, perfluroisobuytlene, phosgene, phosphine, phosphorus pentafluoride, selenium hexafluoride, silicon hexafluoride, silicon tetrachloride, stilbene, disulfur decafluoride, sulfur tetrafluoride, tellurium hexafluoride, tetraethyl pyrophosphate, tetracthyl dithiopyrophosphate, trifluoroacetyl chloride, tungsten hexafluroide, and radon.

Ultrapure water of the disclosure may be prepared by processes known in the art and used as a starting material for generating the compositions and solutions comprising ultrafine bubbles as disclosed herein. The ultrapure water of the disclosure may be prepared by carbon filtration, by slow sand filtration, by reverse osmosis, by electro-deionization treatment, by ultraviolet light exposure, or by a combination comprising two or more of the processes described herein. For example, the ultrapure water of the disclosure may be prepared by a sequential process comprising each of carbon filtration, slow sand filtration, reverse osmosis, electro-deionization treatment, and ultraviolet light exposure. Alternatively, the ultrapure water may be prepared according to one or more of the processes described herein in combination with other methods of water purification known in the art but not expressly recited herein.

The ultrapure water may be prepared by a process comprising the steps of: filtering a volume of water with a carbon filter to produce an amount of water with a low chlorine content; removing ions in the carbon filtered water by a reverse osmosis process to produce a supply of a deionized water; electro-deionizing the supply of the deionized water from the reverse osmosis process to make an ultrapure water supply; testing the resistivity of the ultrapure water to determine if the resistivity of the ultrapure water is between about 17 meg-ohm cm to about 18.2 meg-ohm cm; repeating a process step for preparing the ultrapure water and retesting the resistivity of the ultrapure water until the ultrapure water has a measured resistivity of between about 17 meg-ohm cm to about 18.2 meg-ohm cm; irradiating the supply of the ultrapure water having a measured resistivity of between about 17 meg-ohm cm to about 18.2 meg-ohm cm with ultraviolet light to make a sterilized ultrapure water supply; and storing the sterilized ultrapure water in a stainless steel container until sterilized ultrapure water is needed to be added in the process to make an aqueous composition comprising an aqueous medium with reduced size ultrafine bubbles containing a solute to improve bioavailability of the aqueous composition.

The ultrapure water is purified of contaminants including, for example, organic and inorganic compounds; dissolved and particulate matter; volatile and non-volatile matter, reactive and inert matter; and hydrophilic and hydrophobic matter. Ultrapure water and commonly used term deionized (DI) water are not the same. An ultrapure water system may include three stages: a pretreatment stage to produce purified water, a primary stage to further purify the water, and a polishing stage. The most widely used requirements for ultrapure water quality are documented by ASTM D5127 “Standard Guide for Ultra-Pure Water Used in the Electronics and Semiconductor Industries” and SEMI F63 “Guide for ultrapure water used in semiconductor processing.”

The polishing stage may include continuously treating and recirculating the purified water in order to maintain stable high purity quality of supplied water. Traditionally the resistivity of water serves as an indication of the level of purity of ultrapure water. Deionized (DI) water may have a purity of at least one million ohms-centimeter or one meg-ohm cm. In a preferred embodiment, the ultrapure water quality is at the theoretical maximum of water resistivity (18.18 meg-ohm cm at 25° C.).

The ultrapure water of the disclosure may have a high oxidative reduction potential including, for example, about 140 to about 160 mV. Further, the pH of the ultrapure water may be between about 3 to about 7, preferably about 4 to about 6 and the resistivity of the ultrapure water may be between about 17 to about 18.2 meg-ohm cm.

In some embodiments of the methods disclosed herein, the compositions and solutions used in the methods include ultrafine bubbles comprising or consisting essentially of ultrapure water and gases released from solution in the ultrapure water, wherein the ultrafine bubbles dissolve, surround, and/or stabilize a solute, and wherein the ultrapure water has a high negative oxidative reduction potential. In further embodiments, the oxidative reduction potential of the ultrapure water is about 80 mV to about 100 mV, about 100 mV to about 120 mV, about 120 mV to about 140 mV, or about 140 mV to about 160 mV. In still further embodiments, the pH of the ultrapure water is between about 4 to about 5, about 5 to about 6, or about 6 to about 7.

A non-gaseous solute dissolved in a composition including ultrafine bubbles comprising or consisting essentially of water and gases released from solution in the water may have an approximately round geometry, a flat plate geometry, a cube geometry, a rod-like geometry, a hollow geometry, and/or a semi-hollow geometry. In some embodiments, a solute dissolved in a composition including ultrafine bubbles comprising or consisting essentially of water and gases released from solution in the water may comprise a primary solute, a mixture of a first solute and a second solute, or a plurality of solutes. The solutes may have one or more additional associated solutes, such as a surface coating, as a subsurface coating, or in a complex with other solutes. The solutes may comprise a liquid, a solid, or be a colloidal system with a colloid and a dispersing agent.

In some embodiments of the methods claimed herein, the non-gaseous solute comprises one or more of a cellular detoxification agent, a hydration agent, an anti-inflammatory agent, a neuroprotective agent, a neuromodulatory agent, or an anti-tumorigenic agent. In some embodiments of the methods claimed herein, the one or more solutes is one or more of comprises at least one of an organic chemical, an inorganic chemical, an electrolyte, or an ion of an ionizable salt. In certain embodiments, wherein the non-gaseous solute comprises at least one of a protein, a peptide, a sugar, a lipid, an oligosaccharide, a poly saccharide, a hormone, a pheromone, a nucleic acid, a DNA nucleotide, an RNA nucleotide, a polynucleotide, an RNA polynucleotide, an inter-leukin, PCR enzyme, a polymerase, or a biological cell organelle. In certain embodiments wherein the non-gaseous solute comprises at least one of an organic compound, a small molecule drug, a biologic drug, an antibody, an ultrasound contrast agent, a radiocontrast agent, a solute suitable for human nutrition, or a biotechnology product.

In some embodiments, the solute dissolved within, surrounded by, and/or stabilized by the ultrafine bubbles is an active pharmaceutical ingredient including but not limited to analgesics, antibiotics, antivirals, antifungals, antiseptics and disinfectants, antimalarials, antipyretics, antidepressants, antipsychotics, antiepileptics, anticoagulants, hormones and hormone modulators, cardiovascular agents, antidiabetic agents, vaccines, immunomodulators, enzymes and enzyme modulators, vitamins and minerals, cytostatics and chemotherapeutic agents, gastrointestinal agents.

The ultrafine bubbles comprising or consisting essentially of water and gases released from solution in the water and have a median diameter of between about 2 to about 400 nanometers or comprise about 10 to about 500 molecules of water per ultrafine bubble. In certain embodiments, the ultrafine bubbles have a median diameter of about 1 nanometer, about 2 nanometers, about 3 nanometers, about 4 nanometers, about 5 nanometers, about 6 nanometers, about 7 nanometers, about 8 nanometers, about 9 nanometers, about 10 nanometers, about 11 nanometers, about 12 nanometers, about 13 nanometers, about 14 nanometers, about 15 nanometers, about 16 nanometers, about 17 nanometers, about 18 nanometers, about 19 nanometers, or about 20 nanometers. In other embodiments, the ultrafine bubbles according to the disclosure comprise a median diameter of about 20 nanometers, about 22 nanometers, about 24 nanometers, about 26 nanometers, about 28 nanometers, or about 30 nanometers. In still other embodiments, the ultrafine bubbles according to the disclosure comprise a median diameter of about 35 nanometers, about 40 nanometers, about 45 nanometers, about 50 nanometers, about 60 nanometers, about 70 nanometers, about 80 nanometers, about 90 nanometers, or about 100 nanometers.

In some embodiments, the ultrafine bubble comprises about 20, about 25, about 30, about 35, about 40, about 45, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500 water molecules. In other embodiments, the ultrafine bubble comprises between about 50 and about 100 water molecules, about 100 to about 150 water molecules, about 150 to about 200 water molecules, about 200 to about 250 water molecules, about 250 to about 300 water molecules, about 300 to about 350 water molecules, about 350 to about 400 water molecules, about 400 to about 450 water molecules, or about 450 to about 500 water molecules.

In some embodiments, the ultrafine bubbles fully dissolve, surround, and/or stabilize a non-gaseous solute or substantially dissolve, surround, and/or stabilize a non-gaseous solute (e.g., dissolves, surrounds, and/or stabilizes about 50%, 55%, 60%, 65%, 70%, 80%, 85%, 90%, 95% or more of an individual ion or molecule of the non-gaseous solute).

Those skilled in the art will recognize different ways of measuring a diameter of an ultrafine bubble of the disclosure. In an exemplary method a diameter of an ultrafine bubble is measured using a Malvern Instruments Zetasizer Nano ZSP, which is a high performance system and particularly suitable for the characterization of ultrafine bubbles, solutes, e.g. proteins and other nanoparticles. Optionally, the particle size measurements for the Zetasizer Nano are automated using a NanoSampler. In another exemplary method a diameter of an ultrafine bubble is measured using liquid-cell transmission electron microscopy (TEM). Additionally, the size distribution and concentration of an ultrafine bubble suspension may be measured on a particle-by-particle basis using tunable resistive pulse sensing (TRPS) or electrical zone sensing, using such instruments as the Izon Exoid or the Beckman Coulter Multisizer 4c, respectively.

In some embodiments, the ultrafine bubble and solutes of the disclosure are measured according to the following non-limiting parameters: ultrafine bubble diameter, particle and molecule size, translational diffusion, electrophoretic mobility, zeta potential of particles at high and low concentrations, viscosity and viscoelasticity of protein and polymer solutions, concentration, and/or molecular weight (e.g. kp).

In some embodiments, the ultrafine bubbles comprising or consisting essentially of water and gases released from solution in the water used in such methods are stable for an extended storage period including, for example, a period of years. In some embodiments, the ultrafine bubbles are stable for about 2 years, about 2.5 years, about 3 years, about 4 years, about 5 years, about 6 years, about 7 years, about 8 years, about 9 years, or about 10 years. In some embodiments, the ultrafine bubbles are stable for a period in excess of 10 years.

In some embodiments, the ultrafine bubbles used in embodiments of the methods stabilize, surround, and/or dissolve a non-gaseous solute for a period of years, for example for 2 years, about 2.5 years, about 3 years, about 4 years, about 5 years, about 6 years, about 7 years, about 8 years, about 9 years, or about 10 years. In further embodiments, the ultrafine bubbles dissolve, surround, and/or stabilize a non-gaseous solute for a period in excess of 10 years.

In some embodiments, the compositions or solutions include ultrafine bubbles comprising or consisting essentially of water and gases released from solution in the water, wherein the water has a high negative oxidative reduction potential including, for example, an oxidative reduction potential of about 140 to about 160 mV. In still further embodiments, the pH of the water is between about 4 to about 6. In some embodiments, the water is ultrapure water.

In some embodiments, the disclosure provides compositions or solutions for use in delivering a non-gaseous solute to the interior of a cell. In other embodiments, the disclosure provides compositions or solutions for use in delivering an active pharmaceutical ingredient to the interior of a human cell.

Embodiments of the methods set forth in the disclosure include compositions or solutions wherein a non-gaseous solute is dissolved within, surrounded by, and/or stabilized by ultrafine bubbles comprising or consisting essentially of water and gases released from solution in the water, and has improved bioavailability relative to a composition or a solution where the solute is not dissolved, stabilized, and/or surrounded by ultrafine bubbles comprising or consisting essentially of water and gases released from solution in the water. In some embodiments, the solute dissolved within, surrounded by, and/or stabilized by ultrafine bubbles has improved bioavailability by virtue of its ability to access the interior of a cell. In some embodiments, the solute dissolved within, surrounded by, and/or stabilized by an ultrafine bubble has improved bioavailability by virtue of its ability to access the interior of a human or animal cell. For example, a water may have an impurity or a solute that is typically incapable of passing through a cell membrane, but the solute dissolved within, surrounded by, and/or stabilized by the ultrafine bubbles of the disclosure are able to cross a cell membrane. In some embodiments, a cell membrane may be a plasma membrane, a nuclear membrane, or any other impermeable barrier defining the boundaries of a cell or an organelle within a cell.

In other embodiments of the methods set forth herein, a solute dissolved within, surrounded by, or stabilized by ultrafine bubbles comprising or consisting essentially of water and gases released from solution in the water has improved bioavailability by virtue of its ability to access an intracellular space. In still other embodiments, the solute dissolved within, surrounded by, and/or stabilized by ultrafine bubbles has improved bioavailability by virtue of its ability to access specific human or animal tissue types, such as intestinal epithelial tissue. In yet other embodiments, an ultrafine bubble comprising or consisting essentially of ultrapure water and gases released from solution in the water has improved bioavailability relative to an ultrafine bubble that does not comprise ultrapure water.

In some embodiments, the aqueous compositions including ultrafine bubbles comprising or consisting essentially of water and gases released from solution in the water and dissolved/surrounded/stabilized solutes have improved bioavailability relative to naturally occurring water and dissolved solutes, and relative to compositions including ultrafine bubbles not formed via gaseous cavitation. In some embodiments, the ultrafine bubbles and dissolved/surrounded/stabilized solutes provided herein render an otherwise unavailable solute bioavailable, in which case the disclosure provides improved bioavailability of the solute relative to the undissolved/unsurrounded/unstabilized solute. In other embodiment, the ultrafine bubbles comprising or consisting essentially of water and gases released from solution in the water, wherein the ultrafine bubbles dissolve/surround/stabilize a solute improve bioavailability of the solute by about 10%, about 20%, about 30%, about 40%, about 50%, about 60%, about 70%, about 80%, or about 90% relative to the undissolved/unsurrounded/unstabilized solute. In further embodiments, the ultrafine bubbles comprising or consisting essentially of water and dissolved/surrounded/stabilized solutes improve bioavailability of the solute by about 1%, about 2%, about 3%, about 4%, about 5%, about 6%, about 7%, about 8%, or about 9%.

In some embodiments, the disclosure provides methods for improving the bioavailability of a solute, including, for example, adding the solute to water and dissolving, surrounding, and/or stabilizing the solute with ultrafine bubbles, wherein the ultrafine bubbles have a median diameter between about 2 to about 400 nanometers.

In some embodiments, the compositions having a solute dissolved/surrounded/stabilized within ultrafine bubbles comprising or consisting essentially of water and gases released from solution in the water used in the disclosed methods have improved stability relative to compositions having the undissolved/unsurrounded/unstabilized solute. In some embodiments, the solute dissolved/surrounded/stabilized by ultrafine bubbles with improved stability has an increased half-life, such as an increased solution half-life. In some embodiments, the solute dissolved/surrounded/stabilized by ultrafine bubbles comprising or consisting essentially of water and gases released from solution in the water has improved stability for extended storage periods relative to the undissolved/unsurrounded/unstabilized solute.

In some embodiments, the compositions or solutions including ultrafine bubbles comprising or consisting essentially of water and gases released from solution in the water and a solute dissolved/surrounded/stabilized within the ultrafine bubbles used in the disclosed methods have improved solubility relative to compositions or solutions including the undissolved/unstabilized/unsurrounded solute.

In some embodiments, the solute dissolved/stabilized within an ultrafine bubble normally has limited or no solubility in water but is solubilized when dissolved/stabilized in ultrafine bubbles comprising or consisting essentially of water and gases released from solution in the water. In alternative embodiments, the solute dissolved/stabilized/surrounded by ultrafine bubbles may have low to moderate solubility in water but is solubilized (e.g., completely solubilized) when dissolved/stabilized/surrounded by ultrafine bubbles comprising or consisting essentially of water and gases released from solution in the water as set forth herein.

In some embodiments, a solute of the disclosure further comprises a surface coating applied before or after dissolving/stabilizing/surrounding the solute with ultrafine bubbles comprising or consisting essentially of water and gases released from solution in the water. For biological applications, such as proteins, the surface coating may be polar to give high aqueous solubility and prevent particle aggregation.

### Process for Making Compositions Comprising Ultrafine Bubbles

The present disclosure also provides methods for dissolving/surrounding/stabilizing a solute with ultrafine bubbles comprising or consisting essentially of water.

In some embodiments, the disclosure provides a process for dissolving, surrounding, and/or stabilizing a non-gaseous solute with ultrafine bubbles comprising or consisting essentially of water and gases released from solution in the water, the process comprising: selecting an amount of solute to add to a volume of water; combining the solute and water in a mixing tank to form a blended aqueous composition; pumping the blended aqueous composition at a selected flow rate through a transfer pipe from the mixing tank to a nozzle with one jet opening or a plurality of jet openings inside a hollow cylinder; using the one jet opening or the plurality of jet openings in the nozzle to jet the blended aqueous composition into the hollow cylinder; wherein the selected flow rate creates a vortex of the blended aqueous composition inside the hollow cylinder that dissolve, surround, and/or stabilizes the solutes and reduce sizes of the ultrafine bubbles in the blended aqueous composition. The process according to certain embodiments may further comprise collecting the composition comprising the solute dissolved within, surrounded by, and/or stabilized by the ultrafine bubbles; and using the reduced size ultrafine bubbles dissolving, surrounding, and/or stabilizing the solute to improve the bioavailability of the solute.

In some embodiments, a process is provided for reducing the size of ultrafine bubbles in a solution of water substantially free of dissolved non-gaseous solutes comprising pumping water at a selected flow rate through a transfer pipe to a nozzle with one jet opening or a plurality of jet openings inside a hollow cylinder; using the one jet opening or the plurality of jet openings in the nozzle to jet the blended composition into the hollow cylinder; wherein the selected flow rate creates a vortex of the blended composition inside the hollow cylinder that dissolve, surround, and/or stabilizes the solutes and reduce the size of the ultrafine bubbles in the blended composition.

In another aspect of the invention disclosed herein, a method for producing a composition comprising water and ultrafine bubbles including gases released from solution in the water is provided. The method includes subjecting water to a combination of hydrodynamic cavitation, shear forces, and low pressure/room temperature boiling to produce ultrafine bubbles formed by release of dissolved gases from the water. In some embodiments, the water is selected from DI water, ultrapure water, tap water, groundwater (e.g., well water), surface water, and reverse osmosis water. In particular embodiments, the water is ultrapure water.

The method may comprise one or more (including all) of the following steps: adding water to a tank; pumping the water at a selected flow rate through a transfer pipe from the tank to a nozzle with one jet opening or a plurality of jet openings inside a hollow cylinder; using the one jet opening or the plurality of jet openings in the nozzle to jet the water into the hollow cylinder; wherein the selected flow rate creates a vortex of the water inside the hollow cylinder, thereby subjecting the water to a combination of hydrodynamic cavitation, shear forces, and thin film boiling to produce ultrafine bubbles formed by release of dissolved gases from the water (i.e., gaseous cavitation); collecting the composition comprising the water and ultrafine bubbles; adding a non-gaseous solute (e.g., a plant nutrient) to the composition comprising the water and ultrafine bubbles; and using the ultrafine bubbles of the composition to dissolve, surround, and/or stabilized a non-gaseous solute to improve the bioavailability of the solute.

In some embodiments, a water supply is subjected to a combination of hydrodynamic cavitation, shear forces, and low pressure/room temperature boiling to form ultrafine bubbles, and the formed ultrafine bubbles from the water supply are added to water to form the composition. In some embodiments, a water supply is subjected to processing that forms ultrafine bubbles via gaseous cavitation, and the formed ultrafine bubbles from the water supply are added to water to form a composition as set forth herein. In other embodiments, ultrafine bubbles comprising water and gases released from solution in a first water source are added to a second water source to make compositions as set forth herein. In some embodiments, a non-gaseous solute is added to the composition including the formed ultrafine bubbles and the water supply to dissolve, surround, and/or stabilize the non-gaseous solute with the formed ultrafine bubbles.

The present disclosure also provides methods for dissolving, surrounding, and/or stabilizing a non-gaseous solute with ultrafine bubbles comprising or consisting essentially of water and gases released from solution in water.

In some embodiments, the disclosure provides a process for dissolving, surrounding, and/or stabilizing a non-gaseous solute with ultrafine bubbles comprising or consisting essentially of water and gases released from solution in water, the process comprising: adding a non-gaseous solute (e.g., a plant nutrient) to a composition comprising the water and ultrafine bubbles formed by release of dissolved gases from the water; and using the ultrafine bubbles of the composition to dissolve, surround, and/or stabilized a non-gaseous solute. The process according to certain embodiments may further comprise collecting the composition comprising the non-gaseous solute dissolved within, surrounded by, and/or stabilized by the ultrafine bubbles; and using the reduced size ultrafine bubbles dissolving, surrounding, and/or stabilizing the non-gaseous solute to improve the bioavailability of the solute.

In some embodiments, the disclosure provides a process for dissolving, surrounding, and/or stabilizing a non-gaseous solute with ultrafine bubbles comprising or consisting essentially of water and gases released from solution in water, the process comprising: selecting an amount of solute to add to a volume of water; combining the solute and water in a mixing tank to form a blended aqueous composition; pumping a volume of water at a selected flow rate through a transfer pipe from the mixing tank to a nozzle with one jet opening or a plurality of jet openings inside a hollow cylinder; using the one jet opening or the plurality of jet openings in the nozzle to jet the volume of water into the hollow cylinder; wherein the selected flow rate creates a vortex of the volume of water inside the hollow cylinder that dissolve, surround, and/or stabilizes the solutes and reduce sizes of the ultrafine bubbles in the blended aqueous composition. The process according to certain embodiments may further comprise collecting the composition comprising the solute dissolved within, surrounded by, and/or stabilized by the ultrafine bubbles; and using the reduced size ultrafine bubbles dissolving, surrounding, and/or stabilizing the solute to improve the bioavailability of the solute.

In some embodiments, a process is provided for reducing the size of ultrafine bubbles in a solution of water substantially free of dissolved non-gaseous solutes comprising pumping water at a selected flow rate through a transfer pipe to a nozzle with one jet opening or a plurality of jet openings inside a hollow cylinder; using the one jet opening or the plurality of jet openings in the nozzle to jet the blended composition into the hollow cylinder; wherein the selected flow rate creates a vortex of the blended composition inside the hollow cylinder that reduces the size of the ultrafine bubbles in the blended composition.

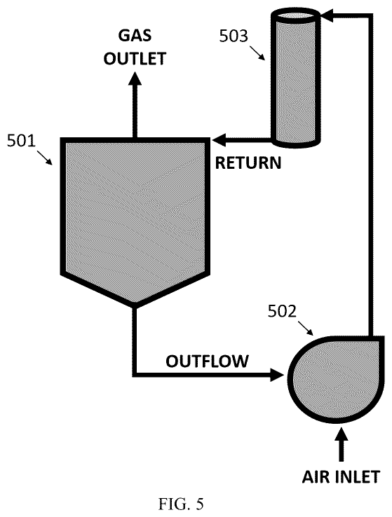

In some embodiments, a process is provided for leveraging the production of microbubbles and nanobubbles together to improve aeration efficiency for fermentation processes in bioreactors. This is accomplished by leveraging the high surface to volume ratio of both microbubbles and ultrafine bubbles, the reduced buoyancy of microbubbles, and the tendency of ultrafine bubbles to effectively deliver nutrients and dissolved gases. The process steps are illustrated in FIG. 5 as follows:

Step. 1: Cell culture media flows (with or without removing suspended cells) from the bioreactor 501 and is enriched with microbubbles via microbubble generator 502 which immediately begin diffusing dissolved oxygen into the media.

Step 2: The microbubble enriched media passes through a hollow cylinder 503, subjecting the media to shear forces to generate ultrafine bubbles from soft cavitation as well as by breaking apart some quantity of microbubbles via shear.

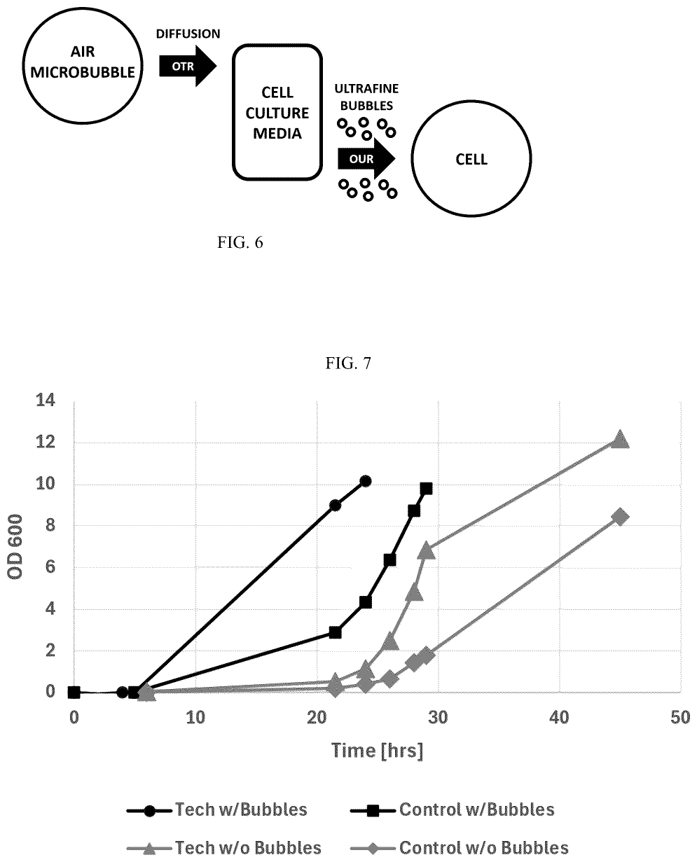

Step 3: The cell culture media containing the remaining microbubbles and ultrafine bubbles are returned to the bioreactor 501, where the microbubbles continue to diffuse dissolved oxygen into the media (oxygen transfer rate-OTR) and the ultrafine bubbles facilitate transport of dissolved oxygen into the cells (oxygen uptake rate-OUR) as illustrated in FIG. 6. The process results in enhanced oxygen delivery in bioreactors by combining microbubbles bubbles for sustained oxygen release and ultrafine bubbles for targeted delivery of dissolved oxygen to cells, respectively.

In some embodiments of the methods, the methods further comprise concentrating the ultrafine bubbles within the composition via rotary evaporation or cross flow filtration. This disclosure is further illustrated by the following examples, which are provided to facilitate the practice of the disclosed methods. These examples do not limit the scope of the disclosure in any way.

In an aspect, a method for increasing biomass of microbes used for fermentative production of compounds is provided. The method includes applying a composition to the microbes, wherein the composition comprises water, at least one non-gaseous solute, and ultrafine bubbles comprising gases released from solution in the water. In some embodiments, the microbes include one or more of Escherichia coli cells, yeast cells, fungal cells, mammalian cells, plant cells, and insect cells. In some embodiments, the at least one non-gaseous solute includes solutes present in microbial growth media. Exemplary growth media for different types of microbes are provided in Table 1. The solutes in the microbial growth media may include at least one of a carbon source (e.g., glucose, sucrose, other sugars and carbohydrates), a nitrogen source (e.g., amino acids, ammonium salts, fetal bovine serum, nitrate, yeast extract, peptones), a phosphorus source (e.g., phosphates), and vitamins (e.g., Complex B, biotin, pantothenic acid, thiamine).

In particular embodiments for E. coli, the at least one non-gaseous solute includes one or more salts. In some embodiments, the salts include MgSO₄ and/or CaCl₂).

In particular embodiments for mammalian cells, the at least one non-gaseous solute includes one or more lipids. In some embodiments, the lipids include essential fatty acids and/or serum lipids. In particular embodiments for mammalian cells, the at least one non-gaseous solute includes one or more growth factors.

In particular embodiments for plant cells, the at least one non-gaseous solute includes one or more plant hormones. In some embodiments, the plant hormones include auxins, cytokinins, and/or other plant growth regulators.

In particular embodiments for insect cells, the at least one non-gaseous solute includes one or more lipids (e.g., essential fatty acids).

TABLE 1
Example growth media for different microbial classes

| Cell Type | Growth Medium |
|---|---|
| E. coli | Luria-Bertani (LB) broth |
| Yeast | Yeast extract-peptone-dextrose (YPD) broth |
| Fungal cells | Sabouraud dextrose broth |
| Mammalian cells | Dulbecco's Modified Eagle Medium (DMEM) |
| Plant cells | Murashige and Skoog (MS) medium |
| Insect cells | Grace's Insect Medium |

In particular embodiments, the microbes include yeast cells. In some embodiments, the yeast cells include one or more of Saccharomyces cerevisiae and Pichia pastoris.

In particular embodiments, the microbes include fungal cells. In some embodiments, the fungal cells include one or more of Aspergillus niger and Trichoderma reesei.

In particular embodiments, the microbes include mammalian cells. In some embodiments, the mammalian cells include one or more of Chinese Hamster Ovary Cells (CHO cells), HEK293 cells, and NSO cells.

In particular embodiments, the microbes include insect cells. In some embodiments, the insect cells include one or more of Sf9 cells and Sf21 cells.

In particular embodiments, the microbes include plant cells. In some embodiments, the plant cells include one or more of Nicotiana benthamiana cells, Arabidopsis thaliana cells, Medicago sativa (Alfalfa) cells, Lycopersicon esculentum (Tomato) cells, Oryza sativa (Rice) cells, Vitis vinifera (Grape) cells.

In an aspect, a method for increasing metabolic rate of microbes used for fermentative production of compounds is provided. The method includes applying a composition to the microbes, wherein the composition comprises water, at least one non-gaseous solute, and ultrafine bubbles comprising gases released from solution in the water. In some embodiments, the at least one non-gaseous solute is a microbe nutrient. In some embodiments, the microbes include one or more of Escherichia coli cells, yeast cells, fungal cells, mammalian cells, plant cells, and insect cells. In particular embodiments, the microbes include one or more of Chinese Hamster Ovary (CHO) Cells, Escherichia coli (E. coli), Yeast Cells (Saccharomyces cerevisiae), NSO Mouse Myeloma Cells, Spodoptera frugiperda (Sf9) Insect Cells, Human Embryonic Kidney 293 (HEK293) Cells, Baby Hamster Kidney (BHK) Cells, Human Cell Lines (e.g., PER.C6, CAP, AGE1.CR). Nicotiana benthamiana Cells, Arabidopsis thaliana Cells, Medicago sativa (Alfalfa) Cells, Lycopersicon esculentum (Tomato) Cells, Oryza sativa (Rice) Cells, and Vitis vinifera (Grape) Cells.

In some embodiments, absorption of one or more microbe nutrients by the microbes after application of the composition is greater in comparison to absorption of the microbe nutrient applied to the microbes by compositions including the microbe nutrient but lacking the ultrafine bubbles. In some embodiments, the microbe nutrients include one or more of amino acids, vitamins, salts and minerals, glucose and other sugars/carbohydrates, hormones and growth factors, serum or serum alternatives, and lipids and fatty acids.

In particular embodiments, the microbes include yeast cells. In some embodiments, the yeast cells include one or more of Saccharomyces cerevisiae and Pichia pastoris.

In particular embodiments, the microbes include fungal cells. In some embodiments, the fungal cells include one or more of Aspergillus niger and Trichoderma reesei.

In particular embodiments, the microbes include mammalian cells. In some embodiments, the mammalian cells include one or more of Chinese Hamster Ovary Cells (CHO cells), HEK293 cells, and NSO cells.

In particular embodiments, the microbes include insect cells. In some embodiments, the insect cells include one or more of Sf9 cells and Sf21 cells.

In an aspect, a method for increasing yields of biological compounds produced via fermentation by microbes is provided. The method comprises applying a composition to the microbes, wherein the composition comprises water, at least one non-gaseous solute, and ultrafine bubbles comprising gases released from solution in the water. In some embodiments, the at least one non-gaseous solute is a microbe nutrient. In some embodiments, the microbe nutrient includes one or more of: amino acids, vitamins, salts and minerals, glucose and other sugars/carbohydrates, hormones and growth factors, serum or serum alternatives, and lipids and fatty acids.

In some embodiments, the biological compounds for which yield is increased include one or more of antibodies, proteins, peptides, enzymes, antibiotics, viral vectors, and metabolites.

In particular embodiments, the biological compounds are antibodies. In some embodiments, the antibodies are monoclonal antibodies. In further embodiments, the monoclonal antibodies have human-like glycosylation patterns.

In particular embodiments, the biological compounds are proteins. In some embodiments, the proteins are recombinant proteins.

In particular embodiments, the biological compounds are metabolites. In some embodiments, the metabolites are secondary metabolites.

In some embodiments, the yields of biological compounds produced via fermentation by microbes after application of the composition comprising water, at least one non-gaseous solute, and ultrafine bubbles comprising gases released from solution in the water is greater in comparison to yields of biological compounds produced via fermentation by microbes to which compositions including the microbe nutrient but lacking the ultrafine bubbles are applied. In particular embodiments, the yields of the biological compounds produced by microbes after application of the composition comprising water, at least one non-gaseous solute, and ultrafine bubbles comprising gases released from solution in the water is increased by at least 1%, by at least 2%, by at least 3%, by at least 4%, by at least 5%, by at least 6%, by at least 7%, by at least 8%, by at least 9%, by at least 10%, by at least 12%, by at least 15%, by at least 17%, by at least 20%, by at least 22%, by at least 25%, by at least 27%, or by at least 30%,

In particular embodiments, the microbes include yeast cells. In some embodiments, the yeast cells include one or more of Saccharomyces cerevisiae and Pichia pastoris.

In particular embodiments, the microbes include fungal cells. In some embodiments, the fungal cells include one or more of Aspergillus niger and Trichoderma reesei.

In particular embodiments, the microbes include mammalian cells. In some embodiments, the mammalian cells include one or more of Chinese Hamster Ovary Cells (CHO cells), HEK293 cells, and NSO cells.

In particular embodiments, the microbes include insect cells. In some embodiments, the insect cells include one or more of Sf9 cells and Sf21 cells.

### EXAMPLES

### Example 1: Methods of Making Compositions Including Water and Ultrafine Bubbles

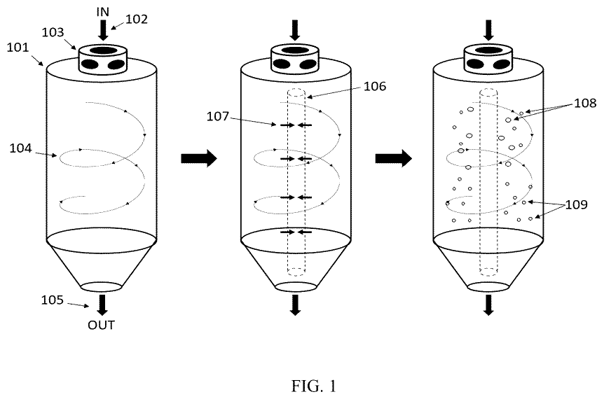

With reference to FIG. 1, a system (101) and a process for making compositions including water and ultrafine bubbles in accordance with embodiments of the disclosure is provided. Water enters the system (101) at step (102) via the nozzle (103) and imparts a vortex flow (104). The vortex core (106) forms as dissolved gases are drawn out of solution due to low pressure at the center (107). Without being bound by theory, it is believed micro- and ultrafine bubbles form (108) spontaneously due to low pressures near to core surface, due to gas being sheared from the core surface, and/or due to room-temperature low pressure boiling at the core surface. Shear and drag forces are believed to break the microbubbles into ultrafine bubbles resulting in a near uniform size distribution (109). The resulting composition including water and ultrafine bubbles flows from the system (101) via the exit (105).

In an embodiment of the disclosure, a non-gaseous solute (e.g., an active pharmaceutical ingredient) is added to the water prior to its entry to the system 101 at step 102, and the resulting composition including water, ultrafine bubbles, and the non-gaseous solute (e.g., an active pharmaceutical ingredient) flows from the system (101) via the exit (105). The ultrafine bubbles of the composition dissolve, surround, and/or stabilize the non-gaseous solute.

In another embodiment of the disclosure, a non-gaseous solute (e.g., an active pharmaceutical ingredient) is added to a composition including water and ultrafine bubbles after the composition exits from the system (101) via the exit (105). The ultrafine bubbles of the composition dissolve, surround, and/or stabilize the non-gaseous solute.

In another embodiment of the disclosure, the system (101) is used to produce an ultrafine bubble suspension or composition comprising water and ultrafine bubbles. The ultrafine bubbles from the ultrafine bubble suspension or composition comprising water and ultrafine bubbles are then added to a different source of water to form a second composition. A non-gaseous solute (e.g., a plant nutrient) is the added to the second composition. The ultrafine bubbles of the second composition dissolve, surround, and/or stabilize the non-gaseous solute.

In another embodiment of the disclosure, the water is “enriched” with microbubbles (bubbles greater than one micron and less than a millimeter in diameter) prior to entering the system (101) via the nozzle (103). These bubbles are added from an exogenous source such as a microbubble generator, venturi, or porous bubbler/membrane in-line or into a tank before processing. The resulting composition exiting via the exit (105) may have higher concentrations of ultrafine bubbles as a result (e.g., greater than 10⁷ ultrafine bubbles/mL). This is due to the breakup of the microbubbles into ultrafine bubbles while passing through the system during which, the microbubbles are exposed to drag forces. Furthermore, by creating microbubbles from specific gases, particularly gases that do not readily dissolve into water (e.g. ozone), the composition of the resulting ultrafine bubbles may be controlled or tailored to include a wider range of gases.

In another embodiment of the disclosure, the water is sparged with one or more specific gases prior to entering the system (101) via the nozzle (103). In some embodiments, the resulting composition of gases contained within the ultrafine bubbles is tailored. For example, when O₂ gas and N₂ gas are sparged or bubbled in water in order to saturate the water prior to undergoing the process within system 101, the resulting composition will have a higher concentration of O₂ and N₂ ultrafine bubbles than if the water had only been exposed to the atmosphere. Such a resulting composition may have particular benefits, such as increased bioavailability and cell permeability when delivering an active pharmaceutical ingredient.

### Example 2: Propidium Iodide Cell Barrier Permeability Study

Propidium iodide (PI) is permeable to dead cells, but impermeable to viable cells. The presence of PI within cells is apparent as PI emits a red fluorescent light in the cells and can serve as a model to study cell permeability. Water compositions were prepared and assessed for ability to penetrate the cell membrane of viable cells in vitro. Three water compositions were prepared with or without PI:
Composition 1: an electro-deionized water with 7 micromolar PI (EDI+PI);
Composition 2: a composition of water with ultrafine bubbles (Investigative Water Lacking PI); and
Composition 3: a composition of water with ultrafine bubbles with 7 micromolar PI (Investigative Water+PI).

Epithelial intestinal, epidermal, tracheal and bronchial epithelial, buccal, nasal, and corneal tissue models were topically exposed with 50 μL of a composition. 5-Chloromethylfluorescein diacetate (CMFDA) was added at the same time as or 30 minutes treatment with the composition. Transepithelial electrical resistance (TEER) measurements were taken before exposure to the composition and 30, 60, and 120 minutes to measure barrier integrity.

At each time point (30, 60, and 120 minutes), tissue samples were rinsed with PBS and used in a MTT viability assay. Samples were also treated with 10% formalin and stained with DAPI for confocal imaging at each time point.

Epithelial intestinal cells treated with Investigative Water+PI showed a reduction of barrier integrity (TEER) to 87.8% and PI uptake after 30 and 120 minutes. Epidermal cells treated with Investigative Water+PI showed a reduction of barrier integrity (TEER) to 91.6% and PI uptake at 30 minutes. Nasal cells treated with Investigative Water+PI showed a reduction of barrier integrity (TEER) to 42.8% and PI uptake at 30 and 60 minutes. Epithelial intestinal, nasal, and epidermal cells showed incorporation of PI into viable cells. Thus, the Investigative Water is capable of delivering organic molecules into viable cells.

Although propidium iodide is not an active pharmaceutical ingredient, this shows that the disclosed composition is able to deliver active pharmaceutical ingredients like complex organic molecules across cell membranes.

### Example 3: Intracellular Zinc Uptake Study

Human embryonic kidney (HEK) cells were treated with a composition prepared in accordance with the disclosure, the composition comprising zinc in a Cellular Hydration Water Advanced Zinc Formulation (Zinc Formulation) to test intracellular zinc uptake in vitro. The composition comprised 93 micromolar zinc (II) ions. The HEK cells were treated with compositions comprising 10%, 50%, or 100% Zinc Formulation. Zinc content was measured at 15, 30, 45, and 60 minutes using a colorimetric assay measuring light absorbance at 540 nm. The changes in intracellular zinc content were compared to a control group of HEK cells treated with a ZnSO₄ solution.

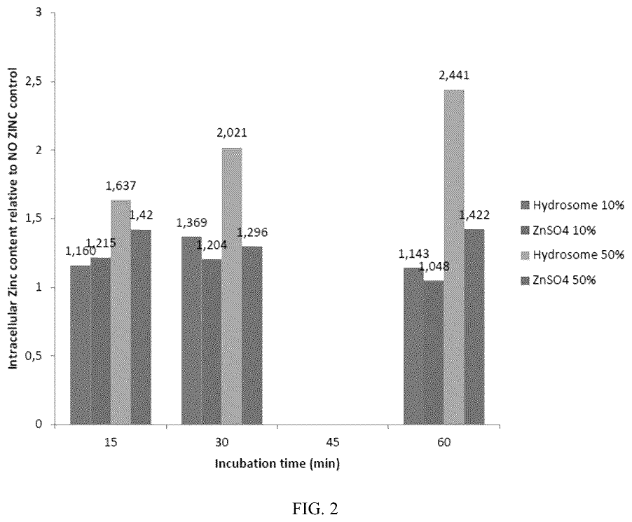

The HEK cells treated with the 10% composition increased intracellular zinc content by 36.9% at 30 minutes; the ZnSO₄ control increased zinc content by 20.4% at the same time point. (FIG. 2) The HEK cells treated with the 50% composition increased intracellular zinc content by 63.7%, 102.1%, and 144.1% at 15 minutes, 30 minutes and 60 minutes, respectively. The ZnSO₄ control increased intracellular zinc content by 42%, 29.6%, and 42.2% at the same respective time points. Thus, the Zinc Formulation is capable of delivering inorganic nutrients into cells in a short period of time.

### Example 4: Improvement of Stem Cell Viability in Ultrafine Bubble (UFB) Suspensions

The effects of the aqueous compositions (also referred to as Hydrosome water) according to the present disclosure in supporting cell viability and proliferation compared to standard cGMP-compliant laboratory-grade water, were studied. The experiment aimed to evaluate whether the present aqueous compositions could improve cell survival and growth dynamics of the TF-1 cell line, a sensitive model for myeloid progenitor cells (bone marrow stem cells), compared to standard cGMP-compliant laboratory-grade water. This cell line is known for its vulnerability, failing to thrive in less-than-ideal conditions, making it an ideal candidate to test the effectiveness of various growth factors and the supportive qualities of different types of water. The research demonstrated that Hydrosome water significantly supports cell survival at high densities where ordinary water fails, suggesting its unique properties are beneficial for stem cell viability and proliferation, especially under suboptimal conditions that typically lead to cell death.

The experimental design involved reconstituting standard cell culture media (RPMI) with the aqueous composition according to the present disclosure (Hydrosome water) and comparing it to media prepared with conventional laboratory-grade water. The study meticulously tested cells at various densities to understand the impact of cell density on growth dynamics, recognizing that stem cells necessitate a minimal density for proliferation. A key aspect of the assay was the use of WST-8, a reagent metabolized by living cells into a colorimetrically detectable compound, facilitating the quantification of viable cells through spectrophotometry. This method allowed for precise monitoring of cell viability over time.

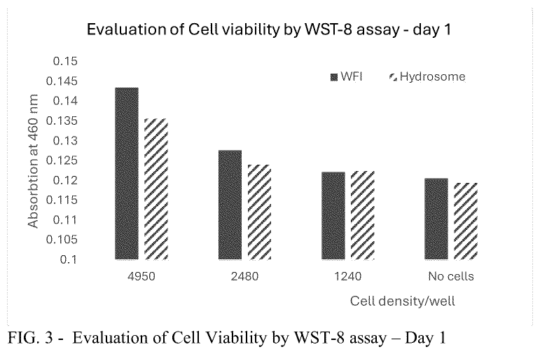

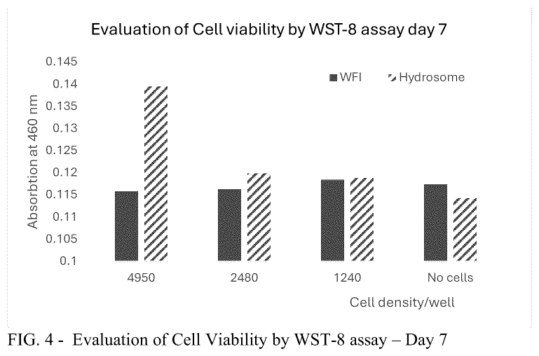

Initial findings demonstrated a proportional relationship between cell viability and density on day 1 across all samples (FIG. 3). However, by day 7, a significant distinction emerged: only the media reconstituted with the water of the present disclosure supported the survival of cells at high densities (FIG. 4). This outcome indicates that present aqueous compositions possess unique properties that enhance the viability of stem cells in conditions where standard laboratory-grade water fails to maintain cellular health.

The example provides compelling evidence of the beneficial effects of the aqueous composition of the present disclosure on stem cell viability, particularly under stressful conditions that typically hinder cell survival. These findings support the use of the water of the present disclosure as a novel solution to improve stem cell culture outcomes, potentially revolutionizing practices in biomedical research and therapeutic applications involving stem cells, particularly with regard to sustaining and enhancing the proliferation and differentiation capabilities of sensitive cell lines like TF-1, setting a new standard for cell culture media reconstitution.

### Example 5: Effects of Gaseous Cavitation on Biomass Fermentation

The present disclosure relates to methods and systems for enhancing the biomass production of microorganisms during fermentation processes. The experiment described in this example aims to decouple the effects of ultrafine bubbles (UFBs) from those caused by the physical process of generating UFBs, particularly focusing on the impacts on Escherichia coli (E. coli) biomass production. The production of UFBs in aqueous solutions has been demonstrated to influence microbial growth and metabolism, yet the individual contributions of UFBs and the physical forces involved in generating them remain unclear. This disclosure seeks to elucidate these factors by conducting controlled fermentation experiments, thus providing insight into the potential applications of UFB technology in industrial microbiology.

### Summary of Experiment

Four parallel E. coli fermentations were conducted to isolate and examine the effects of UFBs and the mechanical processes involved in their generation. The fermentations were carried out in five-gallon fermenters using a chemically defined media. Two of the fermenters were filled with media made using deionized (DI) water, while the other two were filled with media made using UFB-enriched water, which contained a high concentration of UFBs, approximately 10⁹ particles per mL.

Each pair of fermenters (DI media and UFB media) was further divided into two groups: one fermenter in each pair was connected to a recirculation loop containing the present technology, which generates UFBs through a “soft” or gaseous cavitation process, while the other fermenter in each pair was connected to a similar loop without the present technology, serving as a control. The recirculation flow rate and the fermentation conditions were standardized across all experiments, with a temperature of 80° F. and an overpressure of 9 psi.

As shown in FIG. 7, the results of the fermentations demonstrated that the presence of UFBs and the forces generated by the present technology both contribute to increased E. coli The experimental results demonstrate that the use of UFBs in fermentation media significantly reduces fermentation time and enhances E. coli biomass production. The present technology, which generates UFBs through a “soft” cavitation process, independently contributes to a reduction in fermentation time, and a synergistic effect is observed when both UFBs and the present technology are used together, resulting in the most significant reduction in fermentation time and enhanced biomass production growth rates and reduced fermentation times:
The control DI fermentation served as a baseline, with the longest fermentation time.
The DI media utilizing the present technology showed a 33% reduction in fermentation time compared to the control DI, indicating that the forces generated by the technology alone positively impact E. coli growth.
The control fermentation with UFB-enriched media exhibited a 38% reduction in fermentation time compared to the control DI, suggesting that UFBs significantly enhance E. coli growth.
The fermentation utilizing UFB-enriched media and the present technology showed the most substantial reduction in fermentation time, with a 52% decrease compared to the control DI and a 23% improvement over the control with UFBs.

While the present disclosure has been described and illustrated herein by references to various specific materials, procedures, and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims.

1. A composition for delivery of an active pharmaceutical ingredient, the composition comprising:
water;
a non-gaseous solute; and,
ultrafine bubbles comprising gases released from solution in the water, wherein the composition delivers an active pharmaceutical ingredient to a cell.

2. The composition of claim 1, wherein the at least one non-gaseous solute comprises one or more of a cellular detoxification agent, a hydration agent, an anti-inflammatory agent, a neuroprotective agent, a neuromodulatory agent, or an anti-tumorigenic agents.

3. The composition of claim 1, wherein the at least one non-gaseous solute is dissolved within, surrounded by, and/or stabilized by the ultrafine bubbles.

4. The composition of claim 1, wherein the composition increases cell permeability and/or bioavailability of the at least one dissolved non-gaseous solute.

5. The composition of claim 1, wherein the ultrafine bubbles have a median diameter of between 2-400 nanometers.

6. The composition of claim 1, wherein the at least one non-gaseous solute is present at a concentration of about 0.1 wt % to about 95 wt % of the composition.

7. The composition of claim 1, wherein the ultrafine bubbles remain stable within the composition for at least six months.

8. The composition of claim 1, wherein the ultrafine bubbles are concentrated within the composition via rotary evaporation and/or cross flow filtration.

9. The composition according to claim 1, wherein the composition is suitable for oral or sublingual delivery, transdermal delivery, or delivery by inhalation.

10. The composition according to claim 1, wherein the ultrafine bubbles of the composition increase bioavailability of the active pharmaceutical ingredient to the cell.

11. A method of delivering an active pharmaceutical ingredient to a subject, the method comprising:
creating a composition comprising water and ultrafine bubbles comprising gases released from solution in the water;
dissolving the active pharmaceutical ingredient into the composition;
stabilizing the active pharmaceutical ingredient within the ultrafine bubbles;
administering the composition to a subject; and,
wherein the composition increases bioavailability of delivering the active pharmaceutical ingredient to a cell of the subject.

12. The method of delivering an active pharmaceutical ingredient according to claim 11, wherein the active pharmaceutical ingredient is small molecule drug, a protein, a peptide, or a combination thereof.

13. A method of delivering an active pharmaceutical ingredient to a subject, the method comprising the steps of:
creating a composition of ultrafine bubbles comprising gases released from a solution in water:
adding a non-gaseous solute in the form of an active pharmaceutical ingredient to the ultrafine bubble composition;
using the ultrafine bubbles of the composition to encapsulate the non-gaseous solute;
administering the composition of ultrafine bubbles and non-gasous solute combination to at least one cell of the subject.

14. The method according to claim 13, wherein the composition is administered orally, sublingually, transdermally, or by inhalation.

15. The method according to claim 13, wherein the active pharmaceutical ingredient comprises one or more of a cellular detoxification agent, a hydration agent, an anti-inflammatory agent, a neuroprotective agent, a neuromodulatory agent, or an anti-tumorigenic agent.

16. A method for increasing biomass of microbes used for fermentative production of compounds, the method comprising the steps of:
providing composition comprising water, at least one non-gaseous solute, and ultrafine bubbles comprising gases released from solution in the water;
adding a culture of microbes to the composition;
culturing the microbes through fermentation; and,
measuring an increase in biomass production and decrease in fermentation time of the microbes.

17. The method of claim 16, wherein the at least one non-gaseous solute is a microbe nutrient including one or more of: amino acids, vitamins, salts and minerals, glucose and other sugars/carbohydrates, hormones and growth factors, serum or serum alternatives, and lipids and fatty acids.

18. The method of claim 16, wherein the oxygen transfer rate and/or oxygen uptake rate is increased.

19. The method of claim 16, wherein the yield of the product from fermentation is increased.

20. The method of claim 16, wherein the speed of fermentation is increased, or the time required to produce the desired biomass and/or product yield is reduced.
